(19) United States
(12) Patent Application Publication
(10) Pub. No.: US 2015/0283220 A1
(43) Pub. Date: Oct. 8, 2015

(54) METHODS AND COMPOSITIONS FOR THE TREATMENT OF CANCER

(71) Applicant: BAVARIAN NORDIC, INC., Mountain View,, CA (US)

(72) Inventors: Stefanie Mandl, San Francisco, CA (US); Ryan Rountree, San Jose, CA (US); Alain Delcayre, San Jose, CA (US)

(73) Assignee: Bavarian Nordic, Inc., Mountain View, CA (US)

(21) Appl. No.: 14/435,891

(22) PCT Filed: Oct. 16, 2013
(86) PCT No.: PCT/US13/65197

§ 371 Date: Apr. 15, 2015

Related U.S. Application Data
(60) Provisional application No. 61/716,181, filed on Oct. 19, 2012.

(51) Int. Cl. A61K 39/00; C12N 15/86; C12N 7/00

(52) U.S. Cl. CPC A61K 39/0011; C12N 7/00; C12N 15/86; C12N 2710/24134; A61K 2039/5256

(57) ABSTRACT

Provided herein are vaccine compositions having potent adjuvant activity. Such compositions are capable of increasing anti-gen-specific antibody responses and increased frequency of Her-2-specific CD4+ T-cells, have improved anti-tumor efficacy, and are useful in methods of treating cancer.

### FIELD OF THE INVENTION

The invention relates to the treatment of cancers using MVA viruses encoding a tumor-associated antigen, particularly in combination with a recombinant version of the same tumor-associated antigen.

### BACKGROUND OF THE INVENTION

Modified Vaccinia virus Ankara (“MVA”) is a highly attenuated poxvirus related to vaccinia virus, a member of the genus Orthopoxvirus, in the family Poxviridae. MVA was generated by 516 serial passages of the Ankara strain of chorioallantois vaccinia virus (“CVA”) on chicken embryo fibroblasts (for review see Mayr, A., et al. Infection 3, 6-14 (1975)). As a consequence of these long-term passages, the genome of the resulting MVA virus had about 31 kilobases of its genomic sequence deleted and therefore, was described as highly host cell restricted for replication to avian cells (Meyer, H. et al., J. Gen. Virol. 72, 1031-1038 (1991)). It was subsequently shown in a variety of animal models that the resulting MVA was significantly avirulent (Mayr, A. & Danner, K., Der. Biol. Stand. 41: 225-34 (1978)). Additionally, this MVA strain has been tested in clinical trials as a vaccine to immunize against the human smallpox disease (Mayr et al., Zbl. Bakt. Hyg. I, Abt. Org. B 167, 375-390 (1987); Stickl et al., Dtsch. med. Wschr. 99, 2386-2392 (1974)). These studies involved over 120,000 humans, including high-risk patients, and proved that MVA had diminished virulence or infectiousness compared to vaccinia-based vaccines, yet still induced a good specific immune response.

In the following decades, MVA was engineered for use as a viral vector for recombinant gene expression or as a recombinant vaccine (Sutter, G. et al., Vaccine 12: 1032-40 (1994)).

Even though Mayr and colleagues demonstrated during the 1970s that MVA is highly attenuated and avirulent in humans and mammals, certain investigators have reported that MVA is not fully attenuated in mammalian and human cell lines since some level of residual replication might occur in these cells. (Blanchard et al., J. Gen. Virol. 79, 1159-1167 (1998); Carroll & Moss, Virol. 238, 198-211 (1997); Altenberger, U.S. Pat. No. 5,185,146; Ambrosini et al., J. Neurosci. Res. 55(5), 569 (1999)). It is assumed that the results reported in these publications have been obtained with various known strains of MVA, since the viruses used essentially differ in their properties, particularly in their growth behavior in various cell lines. Such residual replication is undesirable for a variety of reasons, including safety concerns in connection with use in humans.

Strains of MVA having enhanced safety profiles for use in the development of safer products, such as vaccines or pharmaceuticals, have been described. See, e.g., U.S. Pat. No. 6,761,893 and U.S. Pat. No. 6,193,752. Such strains are capable of significant reproductive replication in non-human cells and cell lines, especially in chicken embryo fibroblasts (CEF), but are not capable of significant reproductive replication in certain human cell lines known to permit replication with known vaccinia strains. Such cell lines include a human keratinocyte cell line, HaCat (Boukamp et al., J. Cell Biol. 106(3): 761-71 (1988)), a human cervix adenocarcinoma cell line, HeLa (deposited at the American Type Culture Collection, deposit number ATCC No. CCL-2), a human embryo kidney cell line, 293 (deposited at the European Collection of Animal Cell Cultures (“ECACC”) under Accession No. 85120602), and a human bone osteosarcoma cell line, 143B (deposited at ECACC under Accession No. No. 91112502). Such strains are also not capable of significant reproductive replication in vivo, for example, in certain mouse strains, such as the transgenic mouse model AGR. 129, which is severely immune-compromised and highly susceptible to a replicating virus. See U.S. Pat. No. 6,761,893. One such MVA strain and its derivatives and recombinants, referred to as “MVA-BN,” has been described. See U.S. Pat. No. 6,761,893 and U.S. Pat. No. 6,193,752.

MVA and MVA-BN have each been engineered for use as a viral vector for recombinant gene expression or for use as a recombinant vaccine. See, e.g., Sutter, G. et al., Vaccine 12: 1032-40 (1994); U.S. Pat. No. 6,761,893; and U.S. Pat. No. 6,193,752.

Certain approaches to cancer immunotherapy have included vaccination with tumor-associated antigens. In some cases, those approaches have included use of a delivery system to stimulate host immune responses to particular tumor-associated antigens. Those delivery systems have included recombinant viral vectors, some of which have been derived from vaccinia virus. See, e.g., Harrop et al., Front. Biosci. 11:804-817 (2006); Arlen et al., Semin. Oncol. 32:549-555 (2005); Liu et al., Proc. Natl. Acad. Sci. USA 101 (suppl. 2):14567-14571 (2004). For example, an active immunotherapy targeting the HER-2 antigen has been tested for the treatment of HER-2 over-expressing breast cancer. HER-2 is a tumor-associated antigen that is over-expressed in certain types of tumor cells in some patients having different types of cancer, such as breast cancer. Immunization with various HER-2 polypeptides has been used to generate an immune response against tumor cells expressing this antigen, as has vaccination with recombinant modified vaccinia virus Ankara (“MVA”) vectors expressing a modified form of the HER2 protein (i.e., MVA-BN−HER2). See, e.g., Renard et al., J. Immunol. 171:1588-1595 (2003); Mittendorf et al., Cancer 106:2309-2317 (2006); Mandl et al., Cancer Immunol. Immunother. 61(1):19-29 (2012).

Previous work with MVA-BN−HER2 showed that it induced a T_{H}1-biased immune response having both antibody and cellular components. See, e.g., Mandl et al., 2012. Some workers have shown that a balanced immune response including both humoral and cell-mediated components is important for protection from and clearance of a variety of pathogens in the context of infectious disease. See, e.g., Hutchings et al., J. Immunol. 175:599-606 (2005). In cancer, the current dogma regards CD8+ T-cells as the primary effector mechanism for anti-tumor activity. However, the present inventors believe that raising a broader tumor-specific immune response which includes both the innate and multiple arms of the adaptive immune system may be beneficial for the prevention and/or treatment of cancer.

In any case, there is clearly a substantial unmet medical need for additional cancer treatments, including active immunotherapies and cancer vaccines like those described herein.

### SUMMARY

In one aspect, provided herein are compositions comprising a recombinant modified vaccinia virus Ankara (“MVA”) expressing a polypeptide comprising a heterologous tumor-associated antigen and a recombinant protein comprising the heterologous tumor-associated antigen. In certain embodiments, the heterologous tumor-associated antigen comprises an antigen selected from the group consisting of a c-erbB-1 (“EGFr”) antigen, a c-erbB-2 (“HER-2” or “c-Neu”) antigen, a c-erbB-3 antigen and a c-erbB-4 antigen. In certain embodiments, the heterologous tumor-associated antigen comprises a HER-2 antigen. In certain embodiments, the HER-2 antigen comprises SEQ ID NO:1. In certain embodiments, the MVA is MVA-Bavarian Nordic (“MVA-BN”). In certain embodiments, administration of the composition induces an improved antigen-specific CD4+ T-cell response as measured by ELISPOT assay or increased antigen-specific increased antigen-specific antibody titersantibody titers as measured by ELISA, compared to administration of a composition comprising only a recombinant MVA expressing a polypeptide comprising a heterologous tumor-associated antigen. In certain embodiments, the composition further comprises one or more pharmaceutically acceptable diluents, buffers, excipients, or carriers.

In another aspect, provided herein are methods of eliciting an antigen-specific immune response in a human subject comprising: (a) administering to the subject a recombinant modified vaccinia virus Ankara (“MVA”) expressing a polypeptide comprising a heterologous tumor-associated antigen and a recombinant protein comprising the heterologous tumor-associated antigen, thereby eliciting an antigen-specific immune response. In certain embodiments, the heterologous tumor-associated antigen comprises an antigen selected from the group consisting of a c-erbB-1 (“EGFr”) antigen, a c-erbB-2 (“HER-2” or “c-Neu”) antigen, a c-erbB-3 antigen and a c-erbB-4 antigen. In certain embodiments, the heterologous tumor-associated antigen comprises a HER-2 antigen. In certain embodiments, the HER-2 antigen comprises SEQ ID NO:1. In certain embodiments, the MVA is MVA-BN. In certain embodiments, administration of the recombinant MVA and the recombinant protein together induces an improved antigen-specific CD4+ T-cell response as measured by ELISPOT assay or increased antigen-specific antibody titers as measured by ELISA, compared to administration of a composition comprising only a recombinant MVA expressing a polypeptide comprising a heterologous tumor-associated antigen. In certain embodiments, the recombinant MVA and the recombinant are formulated with one or more pharmaceutically acceptable diluents, buffers, excipients, or carriers. In certain embodiments, the human subject has a HER-2 over-expressing cancer. In certain embodiments, the HER-2 over-expressing cancer is selected from the group consisting of breast cancer, colon cancer, lung cancer, gastric cancer, pancreatic cancer, bladder cancer, cervical cancer and ovarian cancer. In certain embodiments, the HER-2 over-expressing cancer is a breast cancer. In certain embodiments, the breast cancer is metastatic breast cancer.

In another aspect, provided herein are methods of treating a human cancer patient comprising: (a) identifying a human cancer patient having a cancer expressing a tumor-associated antigen; (b) administering to the patient a therapeutically effective amount of a recombinant modified vaccinia virus Ankara (“MVA”) expressing a polypeptide comprising the heterologous tumor-associated antigen and a therapeutically effective amount of a recombinant protein comprising the heterologous tumor-associated antigen, thereby treating the cancer. In certain embodiments, the heterologous tumor-associated antigen comprises an antigen selected from the group consisting of a c-erbB-1 (“EGFr”) antigen, a c-erbB-2 (“HER-2” or “c-Neu”) antigen, a c-erbB-3 antigen and a c-erbB-4 antigen. In certain embodiments, the heterologous tumor antigen comprises a HER-2 antigen. In certain embodiments, the HER-2 antigen comprises SEQ ID NO:1. In certain embodiments, the MVA is MVA-BN. In certain embodiments, administration of the recombinant MVA and the recombinant protein together induces an improved CD4+ T-cell response as measured by ELISPOT assay or increased antigen-specific antibody titers as measured by ELISA, compared to administration of a composition comprising only a recombinant MVA expressing a polypeptide comprising a heterologous tumor-associated antigen. In certain embodiments, the recombinant MVA and the recombinant protein are formulated with one or more pharmaceutically acceptable diluents, buffers, excipients, or carriers. In certain embodiments, the human cancer patient has a HER-2 over-expressing cancer. In certain embodiments, the HER-2 over-expressing cancer is selected from the group consisting of breast cancer, colon cancer, lung cancer, gastric cancer, pancreatic cancer, bladder cancer, cervical cancer and ovarian cancer. In certain embodiments, the HER-2 over-expressing cancer is a breast cancer. In certain embodiments, the breast cancer is metastatic breast cancer.

In another aspect, provided herein are kits for treating a human cancer patient, comprising: (a) a recombinant modified vaccinia virus Ankara (“MVA”) expressing a polypeptide comprising a heterologous tumor-associated antigen; (b) a recombinant protein comprising the heterologous tumor-associated antigen; and (c) instructions to administer a therapeutically effective amount of the recombinant MVA and a therapeutically effective amount of the recombinant protein to a human cancer patient having a cancer expressing the heterologous tumor-associated antigen. In certain embodiments, the heterologous tumor-associated antigen comprises an antigen selected from the group consisting of a c-erbB-1 (“EGFr”) antigen, a c-erbB-2 (“HER-2” or “c-Neu”) antigen, a c-erB-3 antigen and a c-erbB-4 antigen. In certain embodiments, the heterologous tumor antigen comprises a HER-2 antigen. In certain embodiments, the HER-2 antigen comprises SEQ ID NO:1. In certain embodiments, the MVA is MVA-BN. In certain embodiments, administration of the recombinant MVA and the recombinant protein together induces an improved antigen-specific CD4+ T-cell response as measured by ELISPOT assay or increased antibody titers as measured by ELISA, compared to administration of a composition comprising only a recombinant MVA expressing a polypeptide comprising a heterologous tumor-associated antigen. In certain embodiments, the recombinant MVA and the recombinant protein are formulated with one or more pharmaceutically acceptable diluents, buffers, excipients, or carriers. In certain embodiments, the human cancer patient has a HER-2 over-expressing cancer. In certain embodiments, the HER-2 over-expressing cancer is selected from the group consisting of breast cancer, colon cancer, lung cancer, gastric cancer, pancreatic cancer, bladder cancer, cervical cancer and ovarian cancer. In certain embodiments, the HER-2 over-expressing cancer is a breast cancer. In certain embodiments, the breast cancer is metastatic breast cancer.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one embodiment of the invention and together with the description, serve to explain the principles of the invention.

### BRIEF DESCRIPTION OF THE DRAWINGS

FIG. 1 presents the results of the experiment described in Example 2. FIG. 1A shows the total anti-HER-2 IgG antibody titers and the ratio of IgG2a to IgG1 isotypes. FIG. 1B shows frequencies of responding T-cells in splenocytes were determined by the IFN-γ ELISPOT assay and the amount of secreted cytokines characteristic of T_{H}1-biased (TNF-α) or T_{H}2-biased (IL-5) CD4+ T-cell responses as measured in the supernatants by the BD™ Cytometric Bead Array (CBA) method.

FIG. 2 presents the results of the experiment described in Example 3, showing that statistically-significant anti-tumor activity requires the presence of both live virus and HER2 protein (MVA-BN+HER2) or expression of the HER2 transgene (MVA-BN−HER2).

FIG. 3 presents the results of the experiment described in Example 3, showing that only treatment with MVA-BN−HER2 induces HER-2 specific CD8+ T-cells in the tumor. FIG. 3 also shows that induction of HER-2-specific CD8+ T-cells requires expression of HER-2 from the viral vector.

FIG. 4 presents the results of the experiment described in Example 3, showing that anti-tumor efficacy induced by use of MVA-BN as an adjuvant may be mediated by HER-2-specific antibodies.

FIG. 5 presents the results of the experiment described in Example 4, showing that HER-2-specific antibody titers significantly increased with addition of HER2 protein, and that the titer of isotype IgG1 increased proportionally more than the titer of isotype IgG2a.

FIG. 6 presents the results of the experiment described in Example 5, showing that the addition of recombinant HER2 protein increased CD4+ T-cell responses (HER2 protein and HER-2 ECD OPL) but not CD8+ T-cell responses (HER-2 p63).

FIG. 7 presents the results of the experiment described in Example 6, which tested the effect of vaccination with MVA-BN−HER2 with or without the addition of recombinant HER2 protein on tumor volume over time in the TUBO mouse model for breast cancer.

FIG. 8 presents the results of the experiment described in Example 6. FIG. 8A depicts tumor volume on day 33 post-challenge and day 39 post challenge for every animal in each group. FIG. 8B shows the results of statistical analysis for each group performed by ANOVA with the Bonferroni adjustment.

FIG. 9 presents the results of the experiment described in Example 6. FIG. 9A presents the various antibody titers measured after vaccination by enzyme-linked immunosorbent assay (“ELISA”). FIG. 9B depicts the ratio of IgG2a to IgG1 measured after vaccination.

FIG. 10 presents the results of the experiment described in Example 6. FIG. 10A shows the T-cell response after restimulation with an overlapping peptide library (“OPL”) covering the full-length HER2 extracellular domain (“ECD”). FIG. 10B shows the T-cell response after restimulation with p63, a CD8+ T-cell epitope derived from HER-2 that is active in BALB/c mice. FIG. 10C shows the T-cell response after restimulation with 104.1 protein, the HER2 extracellular domain (“ECD”) modified to incorporate two T_{H} epitopes derived from tetanus toxin.

FIG. 11 presents the results of the experiment described in Example 7, which tested the effect of vaccination with MVA-BN and MVA-BN−HER2 with or without the addition of recombinant HER2 protein on tumor volume over time in the TUBO mouse model for breast cancer.

FIG. 12 presents the results of the experiment described in Example 7, depicting tumor volume on day 29 post-challenge and day 33 post challenge for every animal in each group.

FIG. 13 shows a Kaplan-Meier survival curve for all groups tested in the experiment described in Example 7.

FIG. 14 presents the results of the experiment described in Example 7, depicting the various antibody titers measured by ELISA after vaccination at the end of the tumor study.

FIG. 15 presents the results of the experiment described in Example 8, which tested the effect of vaccination with MVA-BN and MVA-BN−HER2 with or without the addition of recombinant HER2 protein on tumor volume over time in the TUBO mouse model for breast cancer.

FIG. 16 presents the results of the experiment described in Example 8, depicting the various antibody titers measured after vaccination by ELISA, as well as the ratio of IgG2a titer to IgG1 titer. FIG. 16A shows the various antibody titers measured by ELISA after vaccination. FIG. 16B depicts the ratio of IgG2a to IgG1 measured after vaccination.

FIG. 17 presents the results of the ELISPOT experiment described in Example 8. FIG. 17A shows the T-cell response after restimulation with human p63, a CD8+ T-cell epitope derived from HER-2 that is active in BALB/c mice. FIG. 17B shows the T-cell response after restimulation with rat p63, a CD8+ T-cell epitope derived from HER-2 that is active in BALB/c mice. FIG. 17C shows the T-cell response after restimulation with mouse p63, a CD8+ T-cell epitope derived from HER-2 that is active in BALB/c mice. FIG. 17D shows the T-cell response after restimulation with an OPL covering the full-length HER2 ECD OPL. FIG. 17E shows the T-cell response after restimulation with an OPL covering full-length prostate-specific antigen (“PSA”).

FIG. 18 presents the combined results of the experiments described in Examples 6, 7, and 8, depicting the effect of vaccination with MVA-BN and MVA-BN−HER2 with or without the addition of recombinant HER2 protein on days 21/22, 28/29, 33/34, and 39/40 post-tumor challenge.

### BRIEF DESCRIPTION OF THE SEQUENCES

SEQ ID NO:1 is the amino acid sequence of the HER2 protein extracellular domain including two T_{H}-cell epitopes derived from tetanus toxin.

SEQ ID NO:2 is a nucleotide sequence encoding the amino acid sequence of SEQ ID NO:1.

### DESCRIPTION

In one aspect, provided herein are compositions comprising a recombinant modified vaccinia virus Ankara (“MVA”) expressing a polypeptide comprising a heterologous tumor-associated antigen and a recombinant protein comprising the heterologous tumor-associated antigen for use in eliciting an antigen-specific immune response or to treat cancer. Also contemplated herein are compositions comprising a non-recombinant MVA used in combination with a recombinant protein comprising a heterologous tumor-associated antigen for use in eliciting an antigen-specific immune response or to treat cancer.

The term “recombinant” when applied to a nucleic acid, vector, poxvirus and the like refers to a nucleic acid, vector, or poxvirus made by an artificial combination of two or more otherwise heterologous segments of nucleic acid sequence, or to a nucleic acid, vector or poxvirus comprising such an artificial combination of two or more otherwise heterologous segments of nucleic acid sequence. The artificial combination is most commonly accomplished by the artificial manipulation of isolated segments of nucleic acids, using well-established genetic engineering techniques. When applied to a protein, polypeptide and the like, the term refers to a protein or polypeptide expressed (i.e., transcribed and translated) from a recombinant nucleic acid, vector, poxvirus and the like. For example, the term “recombinant protein” or “recombinant polypeptide” applied to a heterologous tumor-associated antigen refers to a recombinant version of a heterologous tumor-associated antigen expressed from a recombinant nucleic acid, vector, poxvirus and the like and subsequently purified for administration to a human subject, such as a human cancer patient.

Recombinant MVAs are generated by insertion of heterologous sequences into an MVA virus. In certain embodiments, the heterologous nucleic acid sequences are inserted into a non-essential region of the virus genome. In certain embodiments, the heterologous nucleic acid sequences are inserted at one of the naturally occurring deletion site of the MVA genome as described in PCT/EP96/02926. Methods for inserting heterologous sequences into the poxviral genome are known to persons skilled in the applicable art. Recombinant proteins are generated by infecting cells with recombinant MVAs or recombinant expression vectors according to standard procedures well-known to those of ordinary skill in the biological arts.

In vaccinia virus, including MVA, in addition to the TK region, other potentially useful insertion regions include, for example, the Hindlll M fragment. In addition, the intergenic regions of the MVA genome can be used as insertion sites for exogenous proteins (see, e.g., U.S. Pat. No. 7,964,374, which is incorporated herein by reference in its entirety), such as a HER2 protein.

Examples of MVA virus strains useful in the practice of the present invention that have been deposited in compliance with the requirements of the Budapest Treaty on the International Recognition of the Deposit of Microorganisms for the Purposes of Patent Procedure are strains MVA 572, deposited at the European Collection of Animal Cell Cultures (ECACC), Vaccine Research and Production Laboratory, Public Health Laboratory Service, Centre for Applied Microbiology and Research, Porton Down, Salisbury, Wiltshire SP4 0JG, United Kingdom, under the deposit number ECACC 94012707 on Jan. 27, 1994, and MVA 575, deposited under the deposit number ECACC 00120707 on Dec. 7, 2000. MVA-BN, deposited on Aug. 30, 2000, under ECACC deposit number V00083008, and its derivatives, are additional exemplary strains.

Although MVA-BN is preferred for its higher safety (because it is less replication competent), all MVAs are suitable for use with the compositions and methods described herein. In certain embodiments, the recombinant MVA is MVA-BN and its derivatives. A definition of MVA-BN and its derivatives is given in PCT/EP01/13628, which is hereby incorporated herein by reference in its entirety.

In certain embodiments, the tumor-associated antigen is modified to include one or more foreign T_{H} epitopes. Such a cancer immunotherapeutic agent is described herein in a non-limiting example and is referred to as “MVA-BN-mHER2.” As described herein, such cancer immunotherapeutic agents, including, but not limited to MVA-BN-mHER2, are useful for the treatment of cancer. The invention allows for the use of such agents in prime/boost vaccination regimens of humans and other mammals, including immune-compromised patients to elicit an antigen-specific immune response and to treat cancer. In certain embodiments, the induced immune response includes both humoral and cellular immune responses, such as, for example, a T_{H}1 immune response in a pre-existing T_{H}2 environment.

The term “polypeptide” refers to a polymer of two or more amino acids joined to each other by peptide bonds or modified peptide bonds. The amino acids may be naturally occurring as well as non-naturally occurring, or a chemical analogue of a naturally occurring amino acid. As used herein, the term also refers to proteins, i.e. functional biomolecules comprising at least one polypeptide. When comprising at least two polypeptides, polypeptides may form complexes, either covalently or non-covalently linked. The peptides and polypeptides in a protein can be glycosylated and/or lipidated and/or comprise prosthetic groups.

In certain embodiments, the MVA is MVA-BN, deposited under the Budapest Treaty on Aug. 30, 2000, at ECACC under deposit number V00083008, and described in U.S. Pat. No. 6,761,893 and U.S. Pat. No. 6,193,752.

The vaccinia virus MVA was generated by 516 serial passages on chicken embryo fibroblasts of the Ankara strain of Vaccinia virus, referred to as chorioallantois virus Ankara (“CVA”; for review see A. Mayr et al., Infection 3, 6-14 (1975)). The genome of the resulting attenuated MVA lacks approximately 31 kilobase pairs of genomic DNA compared to the parental CVA strain and is highly host-cell restricted to avian cells (H. Meyer et al., J. Gen. Virol. 72, 1031-1038 (1991)). It was shown in a variety of animal models that the resulting MVA was significantly avirulent (A. Mayr & K. Danner Der. Biol. Stand. 41:225-34 (1978)). This MVA strain has been tested in clinical trials as a vaccine to immunize against smallpox in humans (Mayr et al., Zbl. Bakt. Hyg. I, Abt. Org. B 167:375-390 (1987); Stickl et al., Dtsch. Med. Wschr. 99:2386-2392 (1974)). Those studies involved over 120,000 humans, including high-risk patients, and proved that compared to vaccinia virus-based vaccines, MVA had diminished virulence or infectiousness while still able to induce a good specific immune response. Although MVA-BN is preferred for its better safety profile because it is less replication competent than other MVA strains, all MVAs are suitable for this invention, including MVA-BN and its derivatives.

Both MVA and MVA-BN are able to efficiently replicate their DNA in mammalian cells even though they are avirulent. This trait results from the loss of two important host range genes among at least 25 additional mutations and deletions that occurred during its passages in chicken embryo fibroblasts (Meyer et al., Gen. Virol. 72:1031-1038 (1991); Antoine et al., Virol. 244:365-396 (1998)). In contrast to the attenuated Copenhagen strain (NYVAC) and host range restricted avipox (ALVAC), both-early and late transcription in MVA are unimpaired, which allows for continuous gene expression throughout the viral life cycle (Sutter and Moss, Proc. Nat'l Acad. Sci. USA 89:10847-10851 (1992)). In addition, MVA can be used in conditions of pre-existing poxvirus immunity (Ramirez et al., J. Virol. 74:7651-7655 (2000)).

Both MVA and MVA-BN lack approximately 15% (31 kb from six regions) of the genome compared with the ancestral CVA. The deletions affect a number of virulence and host range genes, as well as the gene for Type A inclusion bodies. MVA-BN can attach to and enter human cells where virally-encoded genes are expressed very efficiently. However, assembly and release of progeny virus does not occur. MVA-BN is strongly adapted to primary chicken embryo fibroblast (CEF) cells and does not replicate in human cells. In human cells, viral genes are expressed, and no infectious virus is produced. Despite its high attenuation and reduced virulence, in preclinical studies MVA-BN has been shown to elicit both humoral and cellular immune responses to vaccinia and to heterologous gene products encoded by genes cloned into the MVA genome [E. Harrer et al. (2005), Antivir. Ther. 10(2):285-300; A. Cosma et al. (2003), Vaccine 22(1):21-9; M. DiNicola et al. (2003), Hum. Gene Ther. 14(14):1347-1360; M. DiNicola et al. (2004), Clin. Cancer Res., 10(16):5381-5390].

In certain embodiments, attenuated vaccinia virus strains are useful to induce immune responses in immune-compromised animals, e.g., monkeys infected with SIV (CD4<400/□1 of blood), or immune-compromised humans. The term “immune-compromised” describes the status of the immune system of an individual that exhibits only incomplete immune responses or has a reduced efficiency in the defense against infectious agents.

The reproductive replication of a virus is typically expressed by the amplification ratio. The term “amplification ratio” refers to the ratio of virus produced from an infected cell (“output”) to the amount originally used to infect the cells in the first place (“input”). An amplification ratio of “1” defines an amplification status in which the amount of virus produced from infected cells is the same as the amount initially used to infect the cells, meaning the infected cells are permissive for virus infection and reproduction. An amplification ratio of less than 1 means that infected cells produce less virus than the amount used to infect the cells in the first place, and indicates that the virus lacks the capability of reproductive replication, a measure of virus attenuation.

Thus, the term “not capable of reproductive replication” means that an MVA or MVA derivative has an amplification ratio of less than 1 in one or more human cell lines, such as, for example, the human embryonic kidney 293 cell line (“HEK293”; deposited at ECACC under deposit number ECACC No. 85120602), the human bone osteosarcoma cell line 143B (“143B”; deposited at ECACC under deposit number ECACC No. 91112502), the human cervix adenocarcinoma cell line HeLa (“HeLa”; deposited at the American Type Culture Collection (“ATCC”) under deposit number ATCC No. CCL-2) and the human keratinocyte cell line HaCat (“HaCat”; Boukamp et al., J. Cell Biol. 106(3):761-71 (1988)).

As described in U.S. Pat. No. 6,761,893, U.S. Pat. No. 6,193,752, and International Application No. PCT/EP01/013628, MVA-BN does not reproductively replicate in the human cell lines HEK293, 143B, HeLa and HaCat. For example, in one exemplary experiment, MVA-BN exhibited an amplification ratio of 0.05 to 0.2 in HEK293 cells, an amplification ratio of 0.0 to 0.6 in 143B cells, an amplification ratio of 0.04 to 0.8 in HeLa cells, an amplification ratio of 0.02 to 0.8 in HaCat cells, and an amplification ratio of 0.01 to 0.06 in African green monkey kidney cells (CV1 cells; American Type Culture Collection Deposit Number CCL-70). Thus, MVA-BN does not reproductively replicate in any of the human cell lines HEK293, 143B, HeLa, and HaCat, or in the African green monkey kidney cell line CV1. In contrast, the amplification ratio of MVA-BN is greater than 1 in primary cultures of chicken embryo fibroblast cells (“CEF”) and in baby hamster kidney cells (“BHK”; deposited at ATCC under deposit number ATCC No. CRL-1632), also as described in U.S. Pat. No. 6,761,893, U.S. Pat. No. 6,193,752, and International Application No. PCT/EP01/013628. Therefore MVA-BN can easily be propagated and amplified in CEF primary cultures with an amplification ratio above 500, and in BHK cells with an amplification ratio above 50.

As noted above, all MVAs are suitable for this invention, including MVA-BN and its derivatives. The term “derivatives” refers to viruses showing essentially the same replication characteristics as the strain deposited with ECACC on Aug. 30, 2000, under deposit number V00080038 but showing differences in one or more parts of its genome. Viruses having the same “replication characteristics” as the deposited virus are viruses that replicate with similar amplification ratios as the deposited strain in CEF cells, BHK cells, in the human cell lines HEK293, 143B, HeLa, and HaCat; and that show similar replication characteristics in vivo, as determined, for example, in the AGR129 transgenic mouse model. In certain embodiments, the recombinant MVA is a derivative of MVA-BN. Such “derivatives” include viruses exhibiting essentially the same replication characteristics as the deposited strain (ECACC Deposit Number V00083008), but exhibiting differences in one or more parts of its genome.

In certain embodiments, the MVA is a recombinant vaccinia virus that contains additional nucleotide sequences that are heterologous to the vaccinia virus. In certain embodiments, the heterologous nucleotide sequences encode epitopes that induce a response by the immune system. In certain embodiments, the recombinant MVA is used to vaccinate against the proteins or agents comprising the epitope.

### Exemplary Tumor-Associated Antigens

In certain embodiments, an immune response is produced in a subject against a cell-associated polypeptide antigen. In certain embodiments, the cell-associated polypeptide antigen is a tumor-associated antigen. In certain embodiments, the MVA comprises at least one tumor-associated antigen.

Numerous tumor-associated antigens are known in the art. Exemplary tumor-associated antigens include, but are not limited to, 5-α-reductase, α-fetoprotein, AM-1, APC, April, BAGE, β-catenin, Bcl12, bcr-abl, CA-125, CASP-8/FLICE, Cathepsins, CD19, CD20, CD21, CD23, CD22, CD33 CD35, CD44, CD45, CD46, CD5, CD52, CD55, CD59, CDC27, CDK4, CEA, c-myc, Cox-2, DCC, DcR3, E6/E7, CGFR, EMBP, Dna78, farnesyl transferase, FGF8b, FGF8a, FLK-1/KDR, folic acid receptor, G250, GAGE-family, gastrin 17, gastrin-releasing hormone, GD2/GD3/GM2, GnRH, GnTV, GP1, gp100/Pmel17, gp-100-in4, gp15, gp75/TRP-1, hCG, heparanse, Her2/neu, HMTV, Hsp70, hTERT, IGFR1, IL-13R, iNOS, Ki67, KIAA0205, K-ras, H-ras, N-ras, KSA, LKLR-FUT, MAGE-family, mammaglobin, MAP17, melan-A/MART-1, mesothelin, MIC A/B, MT-MMPs, mucin, NY-ESO-1, osteonectin, p15, P170/MDR1, p53, p97/melanotransferrin, PAI-1, PDGF, μPA, PRAME, probasin, progenipoietin, PSA, PSM, RAGE-1, Rb, RCAS1, SART-1, SSX-family, STAT3, STn, TAG-72, TGF-α, TGF-β, Thymosin-beta-15, TNF-α, TP1, TRP-2, tyrosinase, VEGF, ZAG, p16INK4, and glutathione-S-transferase.

One exemplary tumor-associated antigen is HER-2. HER-2 is a member of the epidermal growth factor receptor family (c-erbB) which consists of four different receptors to date: c-erbB-1 (EGFr), c-erbB-2 (HER-2, c-Neu), c-erbB-3 and c-erbB-4 (Salomon et al., Crit. Rev. Oncol. Heznatol. 19:183-232 (1995)). C-erbB-3 and c-erbB-4 are less well characterized than EGFr and HER-2. HER-2 is an integral membrane glycoprotein. The mature protein has a molecular weight of 185 kD with structural features that closely resemble the EGFr receptor (Prigent et al., Prog. Growth Factor Res. 4(1):1-24 (1992)). EGFr is also an integral membrane receptor consisting of one subunit. It has an apparent molecular weight of 170 kD and consists of a surface ligand-binding domain of 621 amino acids, a single hydrophobic transmembrane domain of 23 amino acids, and a highly conserved cytoplasmic tyrosine kinase domain of 542 amino acids. The protein is N-glycosylated (Prigent et al., EMBO J. 13(12):2831-2841 (1994)).

All proteins in this family are tyrosine kinases. Interaction with the receptor ligand leads to receptor dimerization, which increases the catalytic action of the tyrosine kinase. The proteins within the family are able to homo-and hetero-dimerize, which is important for their activity. The EGFr conveys growth-promoting effects and stimulates uptake of glucose and amino acids by cells (Prigent et al., 1992). HER-2 also conveys growth-promoting signals.

The epidermal growth factor receptor is expressed on normal tissues in low amounts, but it is over-expressed in many types of cancers. EGFr is overexpressed in breast cancers (Chrysogelos et al., Breast Cancer Res. Treat. 31(2-3):227-236 (1994)), glioblastomas (Schlegel et al., J. Neurooncol. 22(3):201-207 (1994)), gastric cancer (Tokunaga et al., Cancer 75(6 Suppl.):1472-1477 (1995)), ovarian cancer (van Dam et al., J. Clin. Pathol. 47(10):914-919 (1994)) and others. HER-2 is also expressed in a few normal human tissues in low amount, most characteristically on secretory epithelia. Over-expression of HER-2 occurs in about 30% of breast, gastric, pancreatic, bladder and ovarian cancers.

The expression of these receptors varies depending on the degree of differentiation of the tumors and the cancer type, e.g., in breast cancer, primary tumors overexpress both receptors; whereas in gastric cancer, the overexpression occurs at a later stage in metastatic tumors (Salomon et al., 1995). The number of overexpressed receptors on carcinoma cells is greater than 10⁶ per cell for several head and neck cancers, as well as for vulva, breast and ovarian cancer lines isolated from patients (Dean et al., Clin. Cancer Res. 4:2545-2550 (1994)).

There are several reasons why members of the EGFr family of receptors present a suitable target for tumor immunotherapy. First, they are overexpressed in many types of cancers, which should direct the immune response towards the tumor and enable such immunotherapies to be useful in treating a wide range of patients. Second, the tumors often express or overexpress the ligands for this family of receptors and some are hypersensitive to the proliferative effects mediated by the ligands. Third, patients with tumors that overexpress growth factor receptors often have a poor prognosis. The overexpression has been closely linked with poor prognosis especially in breast cancer, lung cancer, and bladder cancer and can be associated with invasive/metastatic phenotypes, which are rather insensitive to conventional therapies (Ross and Fletcher, Stem Cells 16(6):413-428 (1994)).

### Modified Tumor-Associated Antigens

In certain embodiments, the cell-associated polypeptide antigen is modified such that a CTL response is induced against a cell which presents epitopes derived from a polypeptide antigen on its surface (i.e., antigen-presenting cells (“APC”)), when presented in association with an MHC Class I molecule on the surface of an APC. In certain embodiments, at least one first foreign T_{H} epitope, when presented, is associated with an MHC Class II molecule on the surface of the APC. In certain embodiments, the cell-associated antigen is a tumor-associated antigen.

Exemplary APCs capable of presenting epitopes include dendritic cells and macrophages. Additional exemplary APCs include any pino- or phagocytizing APC, which is capable of simultaneously presenting 1) CTL epitopes bound to MHC class I molecules and 2) T_{H} epitopes bound to MHC class II molecules.

In certain embodiments, modifications to HER-2 are made such that, after administration to a subject, polyclonal antibodies are elicited that predominantly react with HER-2. Such antibodies could attack and eliminate tumor cells as well as prevent metastatic cells from developing into metastases. The effector mechanism of this anti-tumor effect would be mediated via complement and antibody dependent cellular cytotoxicity. In addition, the induced antibodies could also inhibit cancer cell growth through inhibition of growth factor dependent oligo-dimerization and internalization of the receptors. In certain embodiments, such modified HER-2 polypeptide antigens could induce CTL responses directed against known and/or predicted HER-2 epitopes displayed by the tumor cells.

In certain embodiments, a modified HER-2 polypeptide antigen comprises a CTL epitope of the cell-associated polypeptide antigen and a variation, wherein the variation comprises at least one CTL epitope of a foreign T_{H} epitope. Certain such modified HER-2 polypeptide antigens comprising at least one CTL epitope and a variation comprising at least one CTL epitope of a foreign T_{H} epitope, and methods of producing the same, are described in U.S. Pat. No. 7,005,498 and U.S. Patent Pub. Nos. 2004/0141958 and 2006/0008465.

In certain embodiments, a foreign T_{H} epitope is a naturally-occurring “promiscuous” T-cell epitope. Such promiscuous T-cell epitopes are active in a large proportion of individuals of an animal species or an animal population. In certain embodiments, a vaccine comprises such promiscuous T-cell epitopes. In certain such embodiments, use of promiscuous T-cell epitopes reduces the need for a very large number of different CTL epitopes in the same vaccine. Exemplary promiscuous T-cell epitopes include, but are not limited to, epitopes from tetanus toxin, including but not limited to, the P2 and P30 epitopes, diphtheria toxin, Influenza virus hemagluttinin (HA), and P. falciparum CS antigen.

Additional promiscuous T-cell epitopes include peptides capable of binding a large proportion of HLA-DR molecules encoded by the different HLA-DR genes. See, e.g., WO 98/23635; Southwood et. al., (1998), J. Immunol. 160:3363-3373; Sinigaglia et al., (1988) Nature 336:778-780; Rammensee et al., (1995) Immunogen. 41(4):178-228; Chicz et al., (1993) J. Exp. Med. 178:27-47; Hammer et al., (1993) Cell 74:197-203; and Falk et al., (1994) Immunogen. 39:230-242. The latter reference also addresses HLA-DQ and -DP ligands. All epitopes listed in these references are relevant as candidate natural epitopes as described herein, as are epitopes which share common motifs with these.

In certain embodiments, the promiscuous T-cell epitope is an artificial T-cell epitope which is capable of binding a large proportion of HLA haplotypes. In certain embodiments, the artificial T-cell epitope is a pan-DR epitope peptide (“PADRE”) as described in WO 95/07707. See also Alexander et al., (1994) Immunity 1:751-761.

### Modified HER2 Polypeptide Antigens

Various modified HER-2 polypeptide antigens and methods for producing them are described in U.S. Pat. No. 7,005,498, U.S. Patent Publication Number 2004/0141958 and U.S. Patent Publication Number 2006/0008465, all of which are hereby incorporated herein by reference in their entirety. Those documents describe various modified HER-2 polypeptide antigens comprising one or more promiscuous T-cell epitopes at different positions in the HER-2 polypeptide.

The human HER-2 sequence can be divided into a number of domains based solely on the primary structure of the protein. Those domains are as follows. The extracellular (receptor) domain extends from amino acids 1-654 and contains several subdomains as follows: Domain I (N-terminal domain of mature polypeptide) extends from amino acids 1-173; Domain II (Cysteine rich domain, 24 cysteine residues) extends from amino acids 174-323; Domain III (ligand binding domain in the homologous EGF receptor) extends from amino acids 324-483; and Domain IV (Cysteine rich domain, 20 cysteine residues) extends from amino acids 484-623. The transmembrane residues extend from amino acids 654-675. The intracellular (Kinase) domain extends from amino acids 655-1235 and contains the tyrosine kinase domain, which extends from amino acids 655-1010 (core TK domain extends from 725-992); and the C-terminal domain, which extends from amino acids 1011-1235.

Selection of sites in the amino acid sequence of HER-2 to be displaced by either the P2 or P30 human T helper epitopes is described in U.S. Pat. No. 7,005,498 and U.S. Patent Pub. Nos. 2004/0141958 and 2006/0008465. To summarize, the following parameters were considered: 1. Known and predicted CTL epitopes; 2. Homology to related receptors (EGFR in particular); 3. Conservation of cysteine residues; 4. Predicted loop, α-helix and β-sheet structures; 5. Potential N-glycosylation sites; 6. Prediction of exposed and buried amino acid residues; and 7. Domain organization of the protein.

The CTL epitopes appear to be clustered in domain I, domain III, the TM domain and in two or three “hot spots” in the TK domain. As described in U.S. Pat. No. 7,005,498, U.S. Patent Publication Number 2004/0141958 and U.S. Patent Publication Number 2006/0008465, these should be largely conserved.

Regions with a high degree of homology with other receptors are likely to be structurally important for the “overall” tertiary structure of HER-2, and hence for antibody recognition, whereas regions with low homology possibly can be exchanged with only local alterations of the structure as the consequence. Cysteine residues are often involved in intramolecular disulphide bridge formation and are thus involved in the tertiary structure and should not be changed. Regions predicted to form α-helix or β-sheet structures should be avoided as insertion points of foreign epitopes, as these regions are thought to be involved in folding of the protein.

Potential N-glycosylation sites should be conserved if mannosylation of the protein is desired. Regions predicted (by their hydrophobic properties) to be interior in the molecule preferably should be conserved as these could be involved in the folding. In contrast, solvent exposed regions could serve as candidate positions for insertion of the model T_{H} epitopes P2 and P30. Finally, the domain organization of the protein should be taken into consideration because of its relevance for protein structure and function.

As described in U.S. Pat. No. 7,005,498, U.S. Patent Publication Number 2004/0141958 and U.S. Patent Publication Number 2006/0008465, the focus of the strategy has been to conserve the structure of the extracellular part of HER-2 as much as possible, because this is the part of the protein which is relevant as a target for neutralizing antibodies. By contrast, the intracellular part of native membrane bound HER-2 on the surface of cancer cells is inaccessible for the humoral immune system.

Various exemplary constructs using the P2 and P30 epitopes of tetanus toxin inserted in various domains of HER-2 are provided in U.S. Pat. No. 7,005,498, U.S. Patent Publication Number 2004/0141958 and U.S. Patent Publication Number 2006/0008465. One exemplary modified HER-2 polypeptide antigen, referred to as “mHER2,” comprises the extracellular domains and nine amino acids of the transmembrane domain; the P2 epitope inserted in Domain II between amino acid residues 273 to 287 of the modified HER-2 polypeptide; and the P30 epitope inserted in Domain IV between amino acid residues 655 to 675 of the modified HER-2 polypeptide. SEQ ID NO:2 discloses a nucleotide sequence encoding mHER2, which encodes the amino acid sequence of SEQ ID NO:2.

Recombinant MVA-BN−mHER2

In certain embodiments, recombinant MVA comprising a tumor-associated antigen, e.g., MVA-BN−mHER2, is constructed as follows. The initial virus stock is generated by recombination in cell culture using a cell type permissive for replication, e.g., CEF cells. Cells are both inoculated with an attenuated vaccinia virus, e.g., MVA-BN, and transfected with a recombination plasmid (e.g., pBN146) that encodes the tumor-associated antigen, e.g., mHER2, sequence and flanking regions of the virus genome. In certain embodiments, the plasmid pBN146 contains sequences which are also present in MVA-BN (e.g., the 14L and 15L open reading frames). The mHER2 sequence is inserted between the MVA-BN sequences to allow for recombination into the MVA-BN viral genome. In certain embodiments, the plasmid also contains a selection cassette comprising one or more selection genes to allow for selection of recombinant constructs in CEF cells. In certain embodiments, the recombinant MVA comprises a HER-2 antigen comprising the nucleotide sequence of SEQ ID NO:2, encoding the polypeptide of SEQ ID NO:1.

Simultaneous infection and transfection of cultures allows homologous recombination to occur between the viral genome and the recombination plasmid. Insert-carrying virus is then isolated, characterized, and virus stocks prepared. In certain embodiments, virus is passaged in CEF cell cultures in the absence of selection to allow for loss of the region encoding the selection genes, gpt and EGFP.

Nucleic acids encoding a wild-type or modified HER-2 antigen can be operatively linked to expression control sequences. An expression control sequence operatively linked to a coding sequence is joined such that expression of the coding sequence is achieved under conditions compatible with the expression control sequences. The expression control sequences include, but are not limited to, appropriate promoters, enhancers, transcription terminators, a start codon at the beginning a protein-encoding open reading frame, splicing signals for introns, and in-frame stop codons. Suitable promoters include, but are not limited to, the SV40 early promoter, an RSV promoter, the retrovirus LTR, the adenovirus major late promoter, the human CMV immediate early I promoter, and various poxvirus promoters including, but not limited to the following vaccinia virus or MVA—derived promoters: the 30K promoter, the I3 promoter, the PrS promoter, the Pr7.5K, the 40K promoter, the PrSynIIm promoter, and the PrLE1 promoter.

Additional expression control sequences include, but are not limited to, leader sequences, termination codons, polyadenylation signals and any other sequences necessary for the appropriate transcription and subsequent translation of the nucleic acid sequence encoding the desired recombinant protein (e.g., HER-2) in the desired host system. The poxvirus vector may also contain additional elements necessary for the transfer and subsequent replication of the expression vector containing the nucleic acid sequence in the desired host system. It will further be understood by one skilled in the art that such vectors are easily constructed using conventional methods (Ausubel et al., (1987) in “Current Protocols in Molecular Biology,” John Wiley and Sons, New York, N.Y.) and are commercially available.

### Compositions

In one aspect, provided herein are compositions comprising recombinant MVAs expressing a polypeptide comprising a heterologous tumor-associated antigen and a recombinant protein comprising the heterologous tumor-associated antigen. In certain embodiments, the recombinant MVA is MVA 572. In certain embodiments, the recombinant MVA is MVA 575. In certain embodiments, the recombinant MVA is MVA-Bavarian Nordic (“MVA-BN”). Also contemplated herein are compositions comprising a non-recombinant MVA used in combination with a recombinant protein comprising a heterologous tumor-associated antigen for use in eliciting an antigen-specific immune response or to treat cancer.

In certain embodiments, the heterologous tumor-associated antigen comprises an antigen selected from the group consisting of a c-erbB-1 (“EGFr”) antigen, a c-erbB-2 (“HER-2” or “c-Neu”) antigen, a c-erbB-3 antigen and a c-erbB-4 antigen. In certain embodiments, the tumor-associated antigen comprises a HER-2 antigen. In certain embodiments, the tumor-associated antigen consists essentially of a HER-2 antigen. In certain embodiments, the tumor-associated antigen consists of a HER-2 antigen. In certain embodiments, administration of the composition induces an improved antigen-specific CD4+ T-cell response as measured by ELISPOT assay or increased antigen-specific antibody titers as measured by ELISA, compared to administration of a composition comprising only a recombinant MVA expressing a polypeptide comprising a heterologous tumor-associated antigen.

In certain embodiments, the HER-2 antigen comprises one or more heterologous T_{H}-cell epitopes. In certain embodiments, the HER-2 antigen comprises one heterologous T_{H}-cell epitope. In certain embodiments, the HER-2 antigen comprises two heterologous T_{H}-cell epitopes. In certain embodiments, the HER-2 antigen comprises three, four, five, six, seven, eight, nine, ten or more heterologous T_{H}-cell epitopes. In certain embodiments, the HER-2 antigen consists essentially of one or more heterologous T_{H}-cell epitopes. In certain embodiments, the HER-2 antigen consists essentially of one heterologous T_{H}-cell epitope. In certain embodiments, the HER-2 antigen consists essentially of two heterologous T_{H}-cell epitopes. In certain embodiments, the HER-2 antigen consists essentially of three, four, five, six, seven, eight, nine, ten or more heterologous T_{H}-cell epitopes. In certain embodiments, the HER-2 antigen consists of one or more heterologous T_{H}-cell epitopes. In certain embodiments, the HER-2 antigen consists of one heterologous T_{H}-cell epitope. In certain embodiments, the HER-2 antigen consists of two heterologous T_{H}-cell epitopes. In certain embodiments, the HER-2 antigen consists of three, four, five, six, seven, eight, nine, ten or more heterologous T_{H}-cell epitopes. In certain embodiments, administration of the composition induces an improved antigen-specific CD4+ T-cell response as measured by ELISPOT assay or increased antigen-specific antibody titers as measured by ELISA, compared to administration of a composition comprising only a recombinant MVA expressing a polypeptide comprising a heterologous tumor-associated antigen.

In certain embodiments, the HER-2 antigen comprises the amino acid sequence of SEQ ID NO:1. In certain embodiments, the HER-2 antigen comprises the nucleotide sequence of SEQ ID NO:2, encoding the amino acid sequence of SEQ ID NO:1. In certain embodiments, the HER-2 antigen consists essentially of the amino acid sequence of SEQ ID NO:1. In certain embodiments, the HER-2 antigen consists essentially of the nucleotide sequence of SEQ ID NO:2, encoding the amino acid sequence of SEQ ID NO:1. In certain embodiments, the HER-2 antigen consists of the amino acid sequence of SEQ ID NO:1. In certain embodiments, the HER-2 antigen consists of the nucleotide sequence of SEQ ID NO:2, encoding the amino acid sequence of SEQ ID NO:1. In certain embodiments, administration of the composition induces an improved antigen-specific CD4+ T-cell response as measured by ELISPOT assay or increased antigen-specific antibody titers as measured by ELISA, compared to administration of a composition comprising only a recombinant MVA expressing a polypeptide comprising a heterologous tumor-associated antigen.

In certain embodiments, the recombinant protein comprising the heterologous tumor-associated antigen comprises an antigen selected from the group consisting of a c-erbB-1 (“EGFr”) antigen, a c-erbB-2 (“HER-2” or “c-Neu”) antigen, a c-erbB-3 antigen and a c-erbB-4 antigen. In certain embodiments, the recombinant protein comprising the heterologous tumor-associated antigen comprises a HER-2 antigen. In certain embodiments, the recombinant protein comprising the heterologous tumor-associated antigen consists essentially of a HER-2 antigen. In certain embodiments, the recombinant protein consists of the heterologous tumor-associated antigen comprises a HER-2 antigen. In certain embodiments, administration of the composition induces an improved antigen-specific CD4+ T-cell response as measured by ELISPOT assay or increased antigen-specific antibody titers as measured by ELISA, compared to administration of a composition comprising only a recombinant MVA expressing a polypeptide comprising a heterologous tumor-associated antigen.

In certain embodiments, the HER-2 antigen comprises one or more heterologous T_{H}-cell epitopes. In certain embodiments, the HER-2 antigen comprises one heterologous T_{H}-cell epitope. In certain embodiments, the HER-2 antigen comprises two heterologous T_{H}-cell epitopes. In certain embodiments, the HER-2 antigen comprises three, four, five, six, seven, eight, nine, ten or more heterologous T_{H}-cell epitopes. In certain embodiments, the HER-2 antigen consists essentially of one or more heterologous T_{H}-cell epitopes. In certain embodiments, the HER-2 antigen consists essentially of one heterologous T_{H}-cell epitope. In certain embodiments, the HER-2 antigen consists essentially of two heterologous T_{H}-cell epitopes. In certain embodiments, the HER-2 antigen consists essentially of three, four, five, six, seven, eight, nine, ten or more heterologous T_{H}-cell epitopes. In certain embodiments, the HER-2 antigen consists of one or more heterologous T_{H}-cell epitopes. In certain embodiments, the HER-2 antigen consists of one heterologous T_{H}-cell epitope. In certain embodiments, the HER-2 antigen consists of two heterologous T_{H}-cell epitopes. In certain embodiments, the HER-2 antigen consists of three, four, five, six, seven, eight, nine, ten or more heterologous T_{H}-cell epitopes. In certain embodiments, administration of the composition induces an improved antigen-specific CD4+ T-cell response as measured by ELISPOT assay or increased antigen-specific antibody titers as measured by ELISA, compared to administration of a composition comprising only a recombinant MVA expressing a polypeptide comprising a heterologous tumor-associated antigen.

In certain embodiments, the HER-2 antigen comprises the amino acid sequence of SEQ ID NO:1. In certain embodiments, the HER-2 antigen comprises the nucleotide sequence of SEQ ID NO:2, encoding the amino acid sequence of SEQ ID NO:1. . In certain embodiments, the HER-2 antigen consists essentially of the amino acid sequence of SEQ ID NO:1. In certain embodiments, the HER-2 antigen consists essentially of the nucleotide sequence of SEQ ID NO:2, encoding the amino acid sequence of SEQ ID NO:1. In certain embodiments, the HER-2 antigen consists of the amino acid sequence of SEQ ID NO:1. In certain embodiments, the HER-2 antigen consists of the nucleotide sequence of SEQ ID NO:2, encoding the amino acid sequence of SEQ ID NO:1. In certain embodiments, administration of the composition induces an improved antigen-specific CD4+ T-cell response as measured by ELISPOT assay or increased antigen-specific antibody titers as measured by ELISA, compared to administration of a composition comprising only a recombinant MVA expressing a polypeptide comprising a heterologous tumor-associated antigen.

In certain embodiments, the compositions are pharmaceutical compositions comprising one or more pharmaceutically acceptable and/or approved carriers, additives, antibiotics, preservatives, adjuvants, diluents and/or stabilizers. Such additives include, for example, but not limited to, water, saline, glycerol, ethanol, wetting or emulsifying agents, and pH buffering substances. Exemplary carriers are typically large, slowly metabolized molecules such as proteins, polysaccharides, polylactic acids, polyglycolic acids, polymeric amino acids, amino acid copolymers, lipid aggregates, or the like.

The pharmaceutically acceptable carriers of use are conventional. Remington's Pharmaceutical Sciences, by E. W. Martin, Mack Publishing Co., Easton, Pa., 15th Edition (1975), describes compositions and formulations suitable for pharmaceutical delivery of the recombinant MVAs and recombinant proteins disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

A recombinant tumor-associated antigen such as HER-2 and a recombinant MVA encoding a tumor-associated antigen such as HER-2, can be administered by any means known to one of skill in the art (see, e.g., Banga, A., “Parenteral Controlled Delivery of Therapeutic Peptides and Proteins,” in Therapeutic Peptides and Proteins, Technomic Publishing Co., Inc., Lancaster, Pa., 1995), including either locally or systemically, such as by intramuscular, subcutaneous, intraperitoneal, intravenous injection, as well as by oral, nasal, transdermal or anal administration is contemplated. In certain embodiments, administration is by subcutaneous or intramuscular injection. To extend the time during which the peptide or protein is available to stimulate a response, the peptide or protein can be provided as an implant, an oily injection, or as a particulate system. The particulate system can be a microparticle, a microcapsule, a microsphere, a nanocapsule, or similar particle. (see, e.g., Banga 1995). A particulate carrier based on a synthetic polymer has been shown to act as an adjuvant to enhance the immune response, in addition to providing a controlled release. Aluminum salts can also be used as adjuvants to produce an immune response.

Pharmaceutical compositions comprising a recombinant MVA encoding a tumor-associated antigen such as HER-2 and a recombinant tumor-associated antigen such as HER-2 are thus provided herein. Those compositions are used to generate an immune response, such as for immunotherapy. In certain embodiments, the recombinant tumor-associated antigen is mixed with an adjuvant containing two or more of a stabilizing detergent, a micelle-forming agent, and an oil. Suitable stabilizing detergents, micelle-forming agents, and oils are described in U.S. Pat. No. 5,585,103; U.S. Pat. No. 5,709,860; U.S. Pat. No. 5,270,202; and U.S. Pat. No. 5,695,770, all of which are incorporated herein by reference in their entireties. A stabilizing detergent is any detergent that allows the components of the emulsion to remain as a stable emulsion. Such detergents include polysorbate 80 (TWEEN™) (Sorbitan-mono-9-octadecenoate-poly(oxy-1,2-ethanediyl; manufactured by ICI Americas, Wilmington, Del.), TWEEN 40™, TWEEN 20™, TWEEN 60™, Zwittergent™ 3-12, TEEPOL HB7™, and SPAN 85™. These detergents are usually provided in an amount of approximately 0.05 to 0.5% (v/v), such as at about 0.2% (v/v). A micelle forming agent is an agent which is able to stabilize the emulsion formed with the other components such that a micelle-like structure is formed. Such agents generally cause some irritation at the site of injection in order to recruit macrophages to enhance the cellular response. Examples of such agents include polymer surfactants described by BASF Wyandotte publications, e.g., Schmolka, J. Am. Oil. Chem. Soc. 54:110, 1977, and Hunter et al., J. Immunol 129:1244, 1981, PLURONIC™ L62LF, L101, and L64, PEG1000, and TETRONIC™ 1501, 150R1, 701, 901, 1301, and 130R1. The chemical structures of such agents are well known in the art. In certain embodiments, the agent is chosen to have a hydrophile-lipophile balance (HLB) of between 0 and 2, as defined by Hunter and Bennett, J. Immun. 133:3167, 1984. The agent can be provided in an effective amount, for example between 0.5 and 10% (v/v), or in an amount between 1.25 and 5% (v/v).

The oil included in the compositions is chosen to promote the retention of the antigen in oil-in-water emulsion, such as to provide a vehicle for the desired antigen, and preferably has a melting temperature of less than 65° C. such that emulsion is formed either at room temperature (about 20° C. to 25° C.), or once the temperature of the emulsion is brought down to room temperature. Examples of such oils include squalene, Squalane, EICOSANE™, tetratetracontane, glycerol, and peanut oil or other vegetable oils. In certain embodiments, the oil is provided in an amount between 1 and 10% (v/v), or between 2.5 and 5% (v/v). The oil should be both biodegradable and biocompatible so that the body can break down the oil over time, and so that no adverse effects, such as granulomas, are evident upon use of the oil.

In certain embodiments, the adjuvant is a mixture of stabilizing detergents, micelle-forming agents, and oils available under the name PROVAX® (IDEC Pharmaceuticals, San Diego, Calif.). An adjuvant can also be an immunostimulatory nucleic acid, such as a nucleic acid including a CpG motif, or a biological adjuvant (see above).

Controlled-release parenteral formulations can be made as implants, oily injections, or as particulate systems. For a broad overview of protein delivery systems, see Banga, Therapeutic Peptides and Proteins: Formulation, Processing, and Delivery Systems, Technomic Publishing Company, Inc., Lancaster, Pa., 1995. Particulate systems include microspheres, microparticles, microcapsules, nanocapsules, nanospheres, and nanoparticles. Microcapsules contain the therapeutic protein as a central core. In microspheres, the therapeutic agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly (see Kreuter, Colloidal Drug Delivery Systems, J. Kreuter, ed., Marcel Dekker, Inc., New York, N.Y., pp. 219-342, 1994; Tice & Tabibi, Treatise on Controlled Drug Delivery, A. Kydonieus, ed., Marcel Dekker, Inc. New York, N.Y., pp. 315-339, 1992).

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic proteins are known; use of any technically suitable formulation is contemplated herein and is within the level of ordinary skill in the art (see, e.g., U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

In certain embodiments, a pharmaceutical composition for intravenous administration would include about 0.1 μg to 10 mg of a recombinant tumor-associated antigen such as HER-2 per patient per day. Dosages from 0.1 mg up to about 100 mg per patient per day can be used, particularly if the agent is administered to a secluded site and not into the circulatory or lymph system, such as into a body cavity or into the lumen of an organ. Actual methods for preparing administrable compositions will be known or apparent to those skilled in the art and are described in more detail in such publications as Remington's Pharmaceutical Sciences, 19^{th} Ed., Mack Publishing Company, Easton, Pa., 1995.

For the preparation of vaccines, the MVA can be converted into a physiologically acceptable form. In certain embodiments, such preparation is based on experience in the preparation of poxvirus vaccines used for vaccination against smallpox, as described, for example, in Stickl, H. et al., Dtsch. med. Wschr. 99, 2386-2392 (1974).

An exemplary preparation of a recombinant MVA expressing a tumor-associated antigen follows. Purified virus is stored at −80° C. with a titer of 5×10⁸ TCID₅₀/ml formulated in 10 mM Tris, 140 mM NaCl, pH 7.4. For the preparation of vaccine shots, e.g., 10²-10⁸ particles of the virus can be lyophilized in phosphate-buffered saline (“PBS”) in the presence of 2% peptone and 1% human albumin in an ampoule, preferably a glass ampoule. Alternatively, the vaccine shots can be prepared by stepwise, freeze-drying of the virus in a formulation. In certain embodiments, the formulation contains additional excipients such as mannitol, dextran, sugar, glycine, lactose, polyvinylpyrrolidone, or other additives, such as, including, but not limited to, antioxidants or inert gas, stabilizers or recombinant proteins (e.g., human serum albumin) suitable for in vivo administration. The ampoule is then sealed and can be stored at a suitable temperature, for example, between 4° C. and room temperature for several months. However, as long as no need exists, the ampoule is stored preferably at temperatures below −20° C.

In certain embodiments involving vaccination or therapy, the lyophilisate is dissolved in 0.1 to 0.5 ml of an aqueous solution, preferably physiological saline or Tris buffer, and administered either systemically or locally, i.e., by parenteral, subcutaneous, intravenous, intramuscular, intranasal, intradermal, or any other path of administration known to a skilled practitioner. Optimization of the mode of administration, dose, and number of administrations is within the skill and knowledge of one skilled in the art.

Single or multiple administrations of the compositions can be administered depending on the dosage and frequency as required and tolerated by the subject. In certain embodiments, the dosage is administered once as a bolus. In certain embodiments, the dosage can be applied periodically until the desired therapeutic result is achieved. Generally, the dose is sufficient to treat or ameliorate symptoms or signs of disease without producing unacceptable toxicity to the subject.

### Methods of Eliciting an Immune Response

In another aspect, provided herein are methods of eliciting an antigen-specific immune response in a subject comprising: (a) administering to the subject any of the compositions comprising a recombinant modified vaccinia virus Ankara (“MVA”) expressing a polypeptide comprising a heterologous tumor-associated antigen and a recombinant protein comprising the heterologous tumor-associated antigen, thereby eliciting an antigen-specific immune response. The composition comprises an MVA expressing any of the tumor-associated polypeptides disclosed herein and a recombinant protein comprising any of the heterologous tumor-associated antigens disclosed herein. Also contemplated herein are compositions comprising a non-recombinant MVA used in combination with a recombinant protein comprising any of the heterologous tumor-associated antigens disclosed herein.

In certain embodiments, the method of eliciting an antigen-specific immune response in a subject comprises: (a) administering to the subject a recombinant modified vaccinia virus Ankara (“MVA”) expressing a polypeptide comprising a heterologous tumor-associated antigen and a recombinant protein comprising the heterologous tumor-associated antigen, thereby eliciting an antigen-specific immune response. In certain embodiments, the heterologous tumor-associated antigen comprises an antigen selected from the group consisting of a c-erbB-1 (“EGFr”) antigen, a c-erbB-2 (“HER-2” or “c-Neu”) antigen, a c-erbB-3 antigen and a c-erbB-4 antigen. In certain embodiments, the heterologous tumor-associated antigen comprises a HER-2 antigen. In certain embodiments, the HER-2 antigen comprises SEQ ID NO:1. In certain embodiments, the MVA is MVA-BN. In certain embodiments, administration of the recombinant MVA and the recombinant protein together induces an improved antigen-specific CD4+ T-cell response as measured by ELISPOT, or increased antigen-specific antibody titers as measured by ELISA. In certain embodiments, the recombinant MVA and the recombinant are formulated with any one or more of the various pharmaceutically acceptable diluents, buffers, excipients, or carriers disclosed herein. In certain embodiments, the subject has a HER-2 over-expressing cancer. In certain embodiments, the HER-2 over-expressing cancer is a breast cancer, a colon cancer, a lung cancer, a gastric cancer, a pancreatic cancer, a bladder cancer, a cervical cancer or an ovarian cancer. In certain embodiments, the HER-2 over-expressing cancer is a breast cancer. In certain embodiments, the breast cancer is a metastatic breast cancer. In certain embodiments, the subject is a mammal. In certain embodiments, the mammal is a primate. In certain embodiments, the primate is a human.

The recombinant MVA expressing a polypeptide comprising a HER-2 antigen can be administered either systemically or locally, i.e., by parenteral, subcutaneous, intravenous, intramuscular, intranasal, intradermal, or any other path of administration known to a skilled practitioner. In certain embodiments, a dose of 10⁵⁻10¹⁰ TCID₅₀ of the recombinant MVA is administered to the subject. In certain embodiments, a dose of 10⁷⁻10¹⁰ TCID₅₀ of the recombinant MVA is administered to the subject. In certain embodiments, a dose of 10⁸⁻10¹⁰ TCID₅₀ of the recombinant MVA is administered to the subject. In certain embodiments, a dose of 10⁸ 10⁹ TCID₅₀ of the recombinant MVA is administered to the subject.

In certain embodiments, the recombinant HER-2 antigen can be administered either systemically or locally, i.e., by parenteral, subcutaneous, intravenous, intramuscular, intranasal, intradermal, or any other path of administration known to a skilled practitioner. In certain embodiments, a dose of 0.1 μg to 100 mg of the recombinant HER-2 antigen is administered to the subject. In certain embodiments, a dose of 0.1 μg to 100 mg of the recombinant HER-2 antigen is administered to the subject. In certain embodiments, a dose of 1 μg to 100 mg of the recombinant HER-2 antigen is administered to the subject. In certain embodiments, a dose of 10 μg to 100 mg of the recombinant HER-2 antigen is administered to the subject. In certain embodiments, a dose of 100 μg to 100 mg of the recombinant HER-2 antigen is administered to the subject. In certain embodiments, a dose of 1 mg to 100 mg of the recombinant HER-2 antigen is administered to the subject. In certain embodiments, a dose of 10 mg to 100 mg of the recombinant HER-2 antigen is administered to the subject. In certain embodiments, a dose of 25 mg to 100 mg of the recombinant HER-2 antigen is administered to the subject. In certain embodiments, a dose of 50 mg to 100 mg of the recombinant HER-2 antigen is administered to the subject.

In certain embodiments, the recombinant MVA and the recombinant protein can be administered sequentially. In certain embodiments, the recombinant MVA can be administered first, followed by the recombinant protein. In certain embodiments, the recombinant protein can be administered first, followed by the recombinant MVA. In certain embodiments, the recombinant MVA and the recombinant protein can be administered simultaneously.

In certain embodiments, the composition is administered to the human subject or cancer patient as a prime-boost vaccination. The term “prime-boost vaccination” refers to a vaccination strategy using a first, priming injection of a vaccine targeting a specific antigen followed at intervals by one or more boosting injections of the same vaccine. Prime-boost vaccination may be homologous or heterologous. A homologous prime-boost vaccination uses a vaccine comprising the same immunogen and vector for both the priming injection and the one or more boosting injections. A heterologous prime-boost vaccination uses a vaccine comprising the same immunogen for both the priming injection and the one or more boosting injections but different vectors for the priming injection and the one or more boosting injections. For example, a homologous prime-boost vaccination may use an MVA vector comprising nucleic acids expressing Her-2 for both the priming injection and the one or more boosting injections. In contrast, a heterologous prime-boost vaccination may use an MVA vector comprising nucleic acids expressing Her-2 for the priming injection and a fowlpox vector comprising nucleic acids expressing HER-2 for the one or more boosting injections. Heterologous prime-boost vaccination also encompasses various combinations such as, for example, use of a plasmid encoding an immunogen in the priming injection and use of a poxvirus vector encoding the same immunogen in the one or more boosting injections, or use of a recombinant protein immunogen in the priming injection and use of a plasmid or poxvirus vector encoding the same protein immunogen in the one or more boosting injections.

In certain embodiments, the compositions (i.e., the recombinant MVA and the recombinant protein) are administered to the human cancer patient as a prime-boost vaccination. In certain embodiments, the prime-boost vaccination is a homologous prime-boost vaccination. In certain embodiments, the prime-boost vaccination is a heterologous prime-boost vaccination.

In certain embodiments, the one or more additional therapeutically effective amounts of the compositions disclosed herein (i.e., the one or more boosting vaccinations) are administered at intervals comprising days, weeks or months after administration of the initial therapeutically effective amount of the compositions disclosed herein (i.e., the priming vaccination). In certain embodiments, the one or more additional therapeutically effective amounts of the compositions disclosed herein (i.e., the one or more boosting vaccinations) are administered at intervals of 1, 2, 3, 4, 5, 6, 7 or more days after administration of the initial therapeutically effective amount of the compositions disclosed herein (i.e., the priming vaccination). In certain embodiments, the one or more additional therapeutically effective amounts of the compositions disclosed herein (i.e., the one or more boosting vaccinations) are administered at intervals of 1, 2, 3, 4, 5, 6, 7, 8 or more weeks after administration of the initial therapeutically effective amount of the compositions described herein (i.e., the priming vaccination). In certain embodiments, the one or more additional therapeutically effective amounts of the compositions disclosed herein (i.e., the one or more boosting vaccinations) are administered at intervals of 1, 2, 3, 4, 5, 6, 7, 8, 9, 10, 11, 12 or more months after administration of the initial therapeutically effective amount of the compositions disclosed herein (i.e., the priming vaccination). In certain embodiments, the one or more additional therapeutically effective amounts of the compositions disclosed herein (i.e., the one or more boosting vaccinations) are administered at any combination of intervals after administration of the initial therapeutically effective amount of the compositions disclosed herein (i.e., the priming vaccination) (e.g., 1, 2, 3, 4, 5, 6, 7 or more days, 1, 2, 3, 4, 5, 6, 7, 8 or more weeks, or 1, 2, 3, 4, 5, 6, 7, 8, 9, 10, 11, 12 or more months).

### Methods of Treating Cancer

In another aspect, provided herein are methods of treating a human cancer patient comprising: (a) identifying a human cancer patient having a cancer expressing a tumor-associated antigen; (b) administering to the patient a therapeutically effective amount of a recombinant modified vaccinia virus Ankara (“MVA”) expressing a polypeptide comprising the heterologous tumor-associated antigen and a therapeutically effective amount of a recombinant protein comprising the heterologous tumor-associated antigen, thereby treating the cancer. The composition comprises an MVA expressing any of the tumor-associated polypeptides disclosed herein and a recombinant protein comprising any of the heterologous tumor-associated antigens disclosed herein. Also contemplated herein are compositions comprising a non-recombinant MVA used in combination with a recombinant protein comprising any of the heterologous tumor-associated antigens disclosed herein.

A “therapeutically effective amount” is a quantity of a composition sufficient to achieve a desired therapeutic or clinical effect in a subject being treated. For example, a therapeutically effective amount of a poxviral vector comprising a nucleic acid encoding human HER2 protein operably linked to an expression control sequence would be an amount sufficient to elicit a HER-2-specific immune response, to reduce tumor size or burden, to reduce the number of tumor metastases, to delay progression of a cancer, or to increase overall survival of a patient or population of patients having cancer. A therapeutically effective amount of the poxvirus vectors and compositions comprising the poxvirus vectors described herein is an amount sufficient to raise an immune response to HER-2-overexpressing cells and cells with the potential to overexpress HER-2. The immune response must be of sufficient magnitude to slow the proliferation of HER-2-overexpressing cells and cells with the potential to overexpress HER-2, to inhibit their growth, to reduce a sign or a symptom of the tumor, to provide subjective relief of one or more symptoms associated with the tumor or to provide objectively identifiable improvement in one or more symptoms noted by the attending clinician such as, for example, a reduction in tumor size, a decrease in the number of metastatic lesions, a delay in disease progression, or an increase in overall survival, and the like.

In certain embodiments, the method of treating a human cancer patient comprises: (a) administering to the patient a therapeutically effective amount of a recombinant modified vaccinia virus Ankara (“MVA”) expressing a polypeptide comprising a heterologous tumor-associated antigen and a therapeutically effective amount of a recombinant protein comprising the heterologous tumor-associated antigen. In certain embodiments, the heterologous tumor-associated antigen comprises an antigen selected from the group consisting of a c-erbB-1 (“EGFr”) antigen, a c-erbB-2 (“HER-2” or “c-Neu”) antigen, a c-erbB-3 antigen and a c-erbB-4 antigen. In certain embodiments, the heterologous tumor antigen comprises a HER-2 antigen. In certain embodiments, the HER-2 antigen comprises SEQ ID NO:1. In certain embodiments, the MVA is MVA-BN. In certain embodiments, administration of the recombinant MVA and the recombinant protein together induces an improved antigen-specific CD4+ T-cell response as measured by ELISPOT or by increased antigen-specific antibody titers measured by ELISA. In certain embodiments, the recombinant MVA and the recombinant protein are formulated with one or more pharmaceutically acceptable diluents, buffers, excipients, or carriers. In certain embodiments, the human cancer patient has a HER-2 over-expressing cancer. In certain embodiments, the HER-2 over-expressing cancer is a breast cancer, a colon cancer, a lung cancer, a gastric cancer, a pancreatic cancer, a bladder cancer, a cervical cancer or an ovarian cancer. In certain embodiments, the HER-2 over-expressing cancer is a breast cancer. In certain embodiments, the breast cancer is a metastatic breast cancer.

The recombinant MVA expressing a polypeptide comprising a HER-2 antigen can be administered either systemically or locally, i.e., by parenteral, subcutaneous, intravenous, intramuscular, intranasal, intradermal, or any other path of administration known to a skilled practitioner. In certain embodiments, a dose of 10⁵⁻10¹⁰ TCID₅₀ of the recombinant MVA is administered to the subject. In certain embodiments, a dose of 10⁷⁻10¹⁰ TCID₅₀ of the recombinant MVA is administered to the subject. In certain embodiments, a dose of 10⁸⁻10¹⁰ TCID₅₀ of the recombinant MVA is administered to the subject. In certain embodiments, a dose of 10⁸⁻10⁹ TCID₅₀ of the recombinant MVA is administered to the subject.

In certain embodiments, the recombinant HER-2 antigen can be administered either systemically or locally, i.e., by parenteral, subcutaneous, intravenous, intramuscular, intranasal, intradermal, or any other path of administration known to a skilled practitioner. In certain embodiments, a dose of 0.1 μg to 100 mg of the recombinant HER-2 antigen is administered to the subject. In certain embodiments, a dose of 0.1 μg to 100 mg of the recombinant HER-2 antigen is administered to the subject. In certain embodiments, a dose of 1 μg to 100 mg of the recombinant HER-2 antigen is administered to the subject. In certain embodiments, a dose of 10 μg to 100 mg of the recombinant HER-2 antigen is administered to the subject. In certain embodiments, a dose of 100 μg to 100 mg of the recombinant HER-2 antigen is administered to the subject. In certain embodiments, a dose of 1 mg to 100 mg of the recombinant HER-2 antigen is administered to the subject. In certain embodiments, a dose of 10 mg to 100 mg of the recombinant HER-2 antigen is administered to the subject. In certain embodiments, a dose of 25 mg to 100 mg of the recombinant HER-2 antigen is administered to the subject. In certain embodiments, a dose of 50 mg to 100 mg of the recombinant HER-2 antigen is administered to the subject.

In certain embodiments, the recombinant MVA and the recombinant protein can be administered sequentially. In certain embodiments, the recombinant MVA can be administered first, followed by the recombinant protein. In certain embodiments, the recombinant protein can be administered first, followed by the recombinant MVA. In certain embodiments, the recombinant MVA and the recombinant protein can be administered simultaneously.

In certain embodiments, the compositions (i.e., the recombinant MVA and the recombinant protein) are administered to the human cancer patient as a prime-boost vaccination. In certain embodiments, the prime-boost vaccination is a homologous prime-boost vaccination. In certain embodiments, the prime-boost vaccination is a heterologous prime-boost vaccination.

In certain embodiments, the one or more additional therapeutically effective amounts of the compositions disclosed herein (i.e., the one or more boosting vaccinations) are administered at intervals comprising days, weeks or months after administration of the initial therapeutically effective amount of the compositions disclosed herein (i.e., the priming vaccination). In certain embodiments, the one or more additional therapeutically effective amounts of the compositions disclosed herein (i.e., the one or more boosting vaccinations) are administered at intervals of 1, 2, 3, 4, 5, 6, 7 or more days after administration of the initial therapeutically effective amount of the compositions disclosed herein (i.e., the priming vaccination). In certain embodiments, the one or more additional therapeutically effective amounts of the compositions disclosed herein (i.e., the one or more boosting vaccinations) are administered at intervals of 1, 2, 3, 4, 5, 6, 7, 8 or more weeks after administration of the initial therapeutically effective amount of the compositions described herein (i.e., the priming vaccination). In certain embodiments, the one or more additional therapeutically effective amounts of the compositions disclosed herein (i.e., the one or more boosting vaccinations) are administered at intervals of 1, 2, 3, 4, 5, 6, 7, 8, 9, 10, 11, 12 or more months after administration of the initial therapeutically effective amount of the compositions disclosed herein (i.e., the priming vaccination). In certain embodiments, the one or more additional therapeutically effective amounts of the compositions disclosed herein (i.e., the one or more boosting vaccinations) are administered at any combination of intervals after administration of the initial therapeutically effective amount of the compositions disclosed herein (i.e., the priming vaccination) (e.g., 1, 2, 3, 4, 5, 6, 7 or more days, 1, 2, 3, 4, 5, 6, 7, 8 or more weeks, or 1, 2, 3, 4, 5, 6, 7, 8, 9, 10, 11, 12 or more months).

### Kits

In another aspect, provided herein are kits for treating a human cancer patient, comprising: (a) a recombinant modified vaccinia virus Ankara (“MVA”) expressing a polypeptide comprising a heterologous tumor-associated antigen; (b) a recombinant protein comprising the heterologous tumor-associated antigen; and (c) instructions to administer a therapeutically effective amount of the recombinant MVA and a therapeutically effective amount of the recombinant protein to a human cancer patient having a cancer expressing the heterologous tumor-associated antigen. The compositions comprise an MVA expressing any of the tumor-associated polypeptides disclosed herein and a recombinant protein comprising any of the heterologous tumor-associated antigens disclosed herein. Also contemplated herein are compositions comprising a non-recombinant MVA used in combination with a recombinant protein comprising any of the heterologous tumor-associated antigens disclosed herein.

In certain embodiments, the kits for treating a human cancer patient comprise: (a) a composition comprising a recombinant modified vaccinia virus Ankara (“MVA”) expressing a polypeptide comprising a heterologous tumor-associated antigen; (b) a composition comprising a recombinant protein comprising the heterologous tumor-associated antigen; and (c) instructions to administer a therapeutically effective amount of the composition comprising a recombinant MVA and a therapeutically effective amount of the composition comprising a recombinant protein to a human cancer patient having a cancer expressing the heterologous tumor-associated antigen. In certain embodiments, the heterologous tumor-associated antigen comprises an antigen selected from the group consisting of a c-erbB-1 (“EGFr”) antigen, a c-erbB-2 (“HER-2” or “c-Neu”) antigen, a c-erbB-3 antigen and a c-erbB-4 antigen. In certain embodiments, the heterologous tumor antigen comprises a HER-2 antigen. In certain embodiments, the HER-2 antigen comprises SEQ ID NO:1. In certain embodiments, the MVA is MVA-BN. In certain embodiments, administration of the recombinant MVA and the recombinant protein together induces an improved antigen-specific CD4+ T-cell response as measured by ELISPOT or by increased antigen-specific antibody titers as measured by ELISA. In certain embodiments, the recombinant MVA and the recombinant protein are formulated with one or more pharmaceutically acceptable diluents, buffers, excipients, or carriers. In certain embodiments, the human cancer patient has a HER-2 over-expressing cancer. In certain embodiments, the HER-2 over-expressing cancer is a breast cancer, a colon cancer, a lung cancer, a gastric cancer, a pancreatic cancer, a bladder cancer, a cervical cancer or an ovarian cancer. In certain embodiments, the HER-2 over-expressing cancer is a breast cancer. In certain embodiments, the breast cancer is a metastatic breast cancer.

In certain embodiments, the instructions indicate that the recombinant MVA expressing a polypeptide comprising a HER-2 antigen can be administered either systemically or locally, i.e., by parenteral, subcutaneous, intravenous, intramuscular, intranasal, intradermal, or any other path of administration known to a skilled practitioner. In certain embodiments, the instructions indicate that a dose of 10⁵⁻10¹⁰ TCID₅₀ of the recombinant MVA is administered to the subject. In certain embodiments, the instructions indicate that a dose of 10⁷⁻10¹⁰ TCID₅₀ of the recombinant MVA is administered to the subject. In certain embodiments, the instructions indicate that a dose of 10⁸⁻10 TCID₅₀ of the recombinant MVA is administered to the subject. In certain embodiments, the instructions indicate that a dose of 10⁸⁻10⁹ TCID₅₀ of the recombinant MVA is administered to the subject.

In certain embodiments, the instructions indicate that the recombinant HER-2 antigen can be administered either systemically or locally, i.e., by parenteral, subcutaneous, intravenous, intramuscular, intranasal, intradermal, or any other path of administration known to a skilled practitioner. In certain embodiments, the instructions indicate that a dose of 0.1 μg to 100 mg of the recombinant HER-2 antigen is administered to the subject. In certain embodiments, the instructions indicate that a dose of 0.1 μg to 100 mg of the recombinant HER-2 antigen is administered to the subject. In certain embodiments, the instructions indicate that a dose of 1 μg to 100 mg of the recombinant HER-2 antigen is administered to the subject. In certain embodiments, the instructions indicate that a dose of 10 μg to 100 mg of the recombinant HER-2 antigen is administered to the subject. In certain embodiments, the instructions indicate that a dose of 100 μg to 100 mg of the recombinant HER-2 antigen is administered to the subject. In certain embodiments, the instructions indicate that a dose of 1 mg to 100 mg of the recombinant HER-2 antigen is administered to the subject. In certain embodiments, the instructions indicate that a dose of 10 mg to 100 mg of the recombinant HER-2 antigen is administered to the subject. In certain embodiments, the instructions indicate that a dose of 25 mg to 100 mg of the recombinant HER-2 antigen is administered to the subject. In certain embodiments, the instructions indicate that a dose of 50 mg to 100 mg of the recombinant HER-2 antigen is administered to the subject.

In certain embodiments, the instructions indicate that the recombinant MVA and the recombinant protein can be administered sequentially. In certain embodiments, the instructions indicate that the recombinant MVA can be administered first, followed by the recombinant protein. In certain embodiments, the instructions indicate that the recombinant protein can be administered first, followed by the recombinant MVA. In certain embodiments, the instructions indicate that the recombinant MVA and the recombinant protein can be administered simultaneously.

In certain embodiments, the instructions indicate that the compositions (i.e., the recombinant MVA and the recombinant protein) are administered to the human cancer patient as a prime-boost vaccination. In certain embodiments, the prime-boost vaccination is a homologous prime-boost vaccination. In certain embodiments, the prime-boost vaccination is a heterologous prime-boost vaccination.

In certain embodiments, the instructions indicate that the one or more additional therapeutically effective amounts of the compositions disclosed herein (i.e., the one or more boosting vaccinations) are administered at intervals comprising days, weeks or months after administration of the initial therapeutically effective amount of the compositions disclosed herein (i.e., the priming vaccination). In certain embodiments, the instructions indicate that the one or more additional therapeutically effective amounts of the compositions disclosed herein (i.e., the one or more boosting vaccinations) are administered at intervals of 1, 2, 3, 4, 5, 6, 7 or more days after administration of the initial therapeutically effective amount of the compositions disclosed herein (i.e., the priming vaccination). In certain embodiments, the instructions indicate that the one or more additional therapeutically effective amounts of the compositions disclosed herein (i.e., the one or more boosting vaccinations) are administered at intervals of 1, 2, 3, 4, 5, 6, 7, 8 or more weeks after administration of the initial therapeutically effective amount of the compositions described herein (i.e., the priming vaccination). In certain embodiments, the instructions indicate that the one or more additional therapeutically effective amounts of the compositions disclosed herein (i.e., the one or more boosting vaccinations) are administered at intervals of 1, 2, 3, 4, 5, 6, 7, 8, 9, 10, 11, 12 or more months after administration of the initial therapeutically effective amount of the compositions disclosed herein (i.e., the priming vaccination). In certain embodiments, the instructions indicate that the one or more additional therapeutically effective amounts of the compositions disclosed herein (i.e., the one or more boosting vaccinations) are administered at any combination of intervals after administration of the initial therapeutically effective amount of the compositions disclosed herein (i.e., the priming vaccination)(e.g., 1, 2, 3, 4, 5, 6, 7 or more days, 1, 2, 3, 4, 5, 6, 7, 8 or more weeks, or 1, 2, 3, 4, 5, 6, 7, 8, 9, 10, 11, 12 or more months).

### EXAMPLES

### Example 1

### Materials and Methods

Enzyme-Linked Immunosorbent Assay (“ET ISA”). For MVA ELISAs, frozen virus stock was thawed at room temperature and mixed well before use. MVA-BN at 6×10⁷ TCID₅₀/mL diluted in phosphate-buffered saline (“PBS”) was added to a conical tube and 50 μL virus stock was added to every well in an appropriately-sized microtiter plate. Plates were wrapped in Parafilm® and incubated overnight at 4° C. For HER-2 and other protein ELISAs, 50 μL of the desired protein at a concentration of 1 μg/mL in carbonate buffer (200 mM Na₂CO₃) was added to every well in an appropriately-sized microtiter plate. Plates were wrapped in Parafilm® and incubated either at room temperature for 1 hour or overnight at 4° C. After incubation, wells were emptied and washed twice with 150 μL, PBS.

Before blocking the coated plates, all wells were again washed four times with PBS. After the last wash with PBS, the wash buffer was removed, the plates patted dry on paper towels, and 200 μL of undiluted SuperBlock solution (Fisher Scientific, Pittsburgh, Pa., catalog number PI37516) was added to each well using a multichannel pipette. Plates were incubated with blocking solution at room temperature for at least five minutes.

Serum samples were stored at −80° C. before analysis. Samples were thawed on ice and diluted in Dilution Buffer (10% Superblock in PBST (PBS+Tween-20)) according to standard procedures. Dilutions of positive and negative controls were prepared according to the same procedures; positive control was an antibody; negative control was naïve serum for tumor studies.

Before running ELISAs, blocking buffer was removed from the plates. Next, 50 μl Dilution Buffer was added to all rows except for the first row (i.e., rows B-H). Samples and controls were added to the first row (i.e., row A) as follows: A1: 100 μL of diluted positive control antibody; A2: 100 μL of diluted negative control serum; the remaining wells in the first row received 100 μL of diluted serum samples in duplicate. Samples were serially diluted from row A to row H using a multichannel pipette. Plates were then covered with an empty microtiter plate and incubated at room temperature for an hour.

After incubation with the primary antibody, wells were washed four times each with 50 μL wash buffer and then incubated with secondary antibody. Secondary antibody was prepared either by diluting an anti-mouse IgG antibody mixture 1:5000 in dilution buffer or an anti-rabbit IgG antibody 1:2000 in dilution buffer. For ELISAs measuring IgG ratios, the following strain-specific secondary antibodies were used: IgG2a/IgG1 for BALB/c and IgG2c/IgG1 for C57BL/6. 50 μL aliquots of secondary antibody were added per well using a multi-channel pipette. Plates were stacked, covered with an empty microtiter plate, and incubated at room temperature for exactly 1 hour.

At the end of the incubation, plates were washed again four times with PBS, and 100 μL of 3, 3′, 5, 5′-tetramethylbenzidine (“TMB”) was added to each well. Plates were then incubated in the dark at room temperature until the solution turned blue—typically 3-5 minutes. The reaction was stopped by the addition of 100 μL of H₂SO₄ to all wells. Plates were then read on a Multiskan plate reader set to a wavelength of 450 nm.

Enyme-Linked Immunosorbent Spot Assay (“ELISPOT”). All steps prior to developing the assays were done under sterile conditions, preferably in a laminar flow hood.

Using a microtiter plate of the desired size, the membrane of each well was pre-wet with 35 μL of freshly prepared 50% (v/v) ethanol using a multichannel pipette. After a minute, ethanol was removed from the plate and wells were washed with 200 μL of sterile PBS. The desired antibody was diluted in sterile PBS to a concentration of 5 μg/mL and briefly mixed. The wells of each plate were then coated with 50 μL per well of the appropriate antibody using a multichannel pipette. The plates were then covered with the provided lids, stacked if appropriate, wrapped in Parafilm® to prevent them from drying out, and incubated overnight at 4° C.

The next day, the coating antibody was removed from the wells of each plate, and the plates were washed twice with 200 μL of sterile PBS; the second wash was left on the plates until all were ready for blocking. Plates were then blocked by the addition of 100 μL per well of filtered RPMI-10 medium (RPMI-10 is sterile-filtered RPMI-1640 medium supplemented with 10% (v/v) fetal bovine serum (“FBS”), 1% (v/v) penicillin/streptomycin, and 0.5 mM β-Mercaptoethanol; VWR/Mediatech, Brisbane, Calif., catalog no. 16777-180), covered with lids, and placed in an incubator set at 37° C.+5% CO₂ for at least 1 hour.

Protein, peptide, ConA (lectin used as positive control), and virus solutions used for restimulation were diluted in RPMI-10. Each restimulation solution was added to a deep-well 96-well microtiter plate according to the plate map. Plates were then covered with plate sealer and placed at 4° C. until ready for use. Splenocytes were prepared according to standard procedures.

The blocking RPMI-10 solution was removed from the wells, and the plates were then patted dry. Plates were then labeled appropriately to identify the study number, date, group, pool or depletion, and with a red sticker to identify it as an R&D experiment. Using a multichannel pipette, 50 μL of restimulation media was transferred from the appropriate rows of the deep well 96-well plate to the corresponding row of wells on the ELISPOT plate, moving from low concentration to high concentration in each restimulation agent. The splenocytes to be added to each ELISPOT plate were mixed by gently inverting the conical tube of cells several times and then transferred to a sterile v-bottomed boat to ensure an even distribution of cells. Next, 50 μL of splenocytes were added to each well (5×10⁵ cells per well), and the plates were incubated undisturbed in an incubator set at 37° C.+5% CO₂ for ˜40 hours.

Spots were detected in a Laminar Air Flow hood. First, cells were removed from the wells and the plates were patted dry. Wells then were washed twice with 200 μL of PBS, then twice with PBST. The latter two washes were performed simply by immersing each microtiter plate in a bucket containing PBST. This step was done outside the Laminar Air Flow hood. The last wash was left in the wells until the next step in the detection process was initiated.

The appropriate detection antibody (i.e., a biotinylated anti-mouse IFN-γ antibody) was diluted to the desired concentration in PBSTB (PBS+0.1% (v/v) Tween-20+0.5% (w/v) BSA). After diluting the desired detection antibody, the antibody solution was mixed and filtered through a 0.2 μm pore filter. The wash buffer was then removed from each plate and 50 μL of the diluted detecting antibody was added to each well. The plates were then covered with lids and incubated at room temperature for 1 hour. The detecting antibody solution was then removed from the plates, which were then washed by immersing the plates into a bucket containing PBST. Again, the last wash was left in the wells until the next step in the detection process was initiated.

Alkaline phosphatase-conjugated streptavidin (“Streptavidin-AP”) was diluted to an appropriate concentration in PBSTB and filtered through a 0.2 μm steriflip filter. The wash buffer was then removed from each plate and 50 μL of the diluted Streptavidin-AP solution was added to each well. The plates were then covered and incubated at room temperature for 1 hour. The Streptavidin-AP solution was removed from the plates, which were then washed six times by immersing plate into a bucket containing PBST. The last wash was left in the wells until the next step in the detection process was initiated.

The Vector Blue AP Substrate mix (Vector Laboratories, Burlingame, Calif., catalog no. SK-5300) was prepared according to manufacturer's instructions and filtered through a 0.2 μm filter. Next, 50 μL of the Vector Blue AP Substrate mix was added to each well. Plates were then covered with foil. The detection reaction was allowed to proceed for 20-30 minutes, when it was stopped by rinsing each plate thoroughly under tap water. The plate backings were then removed, the bottom of the wells rinsed, and the plates air dried at 4° C. Finally, plates were scanned and spots counted after they were dried.

### Example 2

### MVA-BN Acts as a Potent T_{H}1 Adjuvant for Vaccination with Recombinant HER2 Protein

Groups of BALB/c and C57BL/6 mice (n=5) were vaccinated by subcutaneous injection every two weeks for four cycles (i.e., a total treatment time of eight weeks) with: (1) TBS; (2) 5 μg HER2 formulated in TBS (HER2); (3) 5×10⁷ infectious units (“IU”) MVA-BN+5 μg HER2 formulated in TBS; or (4) 5×10⁷ IU MVA-BN−HER2.

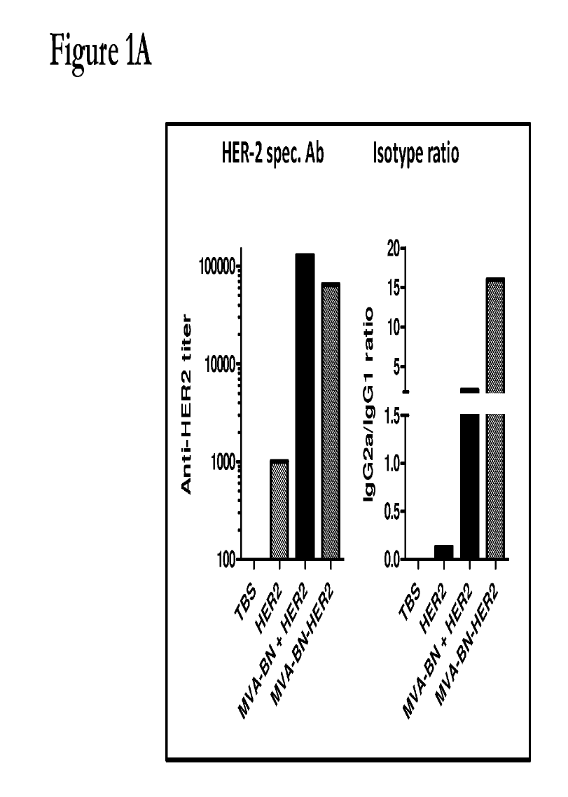

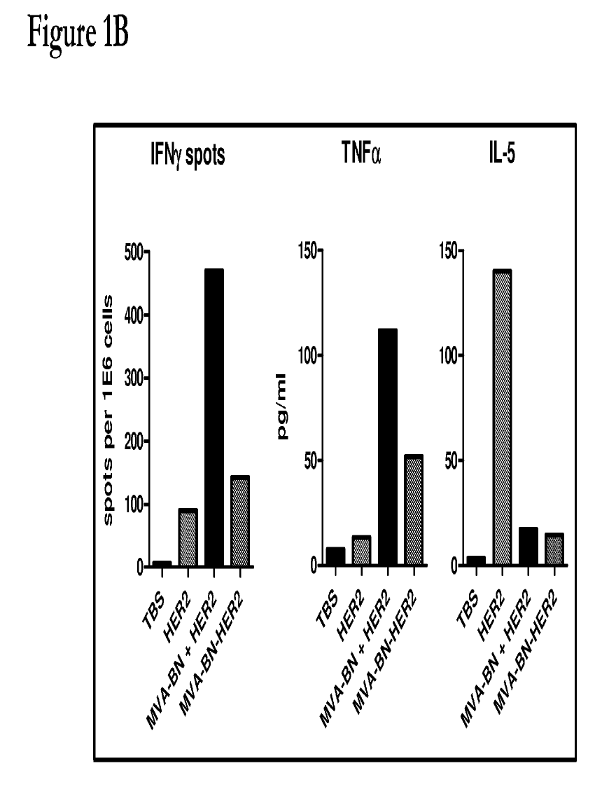

Total immunoglobulin G (“IgG”) titers or IgG isotype ratios (IgG2a to IgG1) were determined on pooled serum samples by ELISAs performed according to the standard protocol described in Example 1. See FIG. 1A. Cellular responses were assessed seven days after the final vaccination by ELISPOT assay performed according to the standard protocol described in Example 1. The frequencies of responding T-cells in splenocytes were determined by the IFN-γ ELISPOT assay. In addition, the amount of secreted cytokines characteristic of T_{H}1-biased (TNF-α) or T_{H}2-biased (IL-5) CD4+ T-cell responses was measured in the supernatants of the same wells by the BD™ Cytometric Bead Array (CBA). See FIG. 1B.

As shown in FIG. 1A, vaccination with either MVA-BN+HER2 protein or MVA-BN−HER2 produced much higher titers of HER2-specific antibodies compared to vaccination with recombinant HER2 protein alone. Similarly, T_{H}1-biased IgG2a:IgG1 ratios were observed after vaccination with either MVA-BN+HER2 protein or MVA-BN−HER2 but not with recombinant HER2 protein alone. See FIG. 1A. ELISPOT and cytokine analysis confirmed these observations: vaccination with either MVA-BN+HER2 protein or MVA-BN−HER2 produced more IFN-γ-secreting spots and higher levels of TNF-α, a cytokine characteristic of T_{H}1-biased CD4+ T-cell responses, while vaccination with recombinant HER2 protein alone produced fewer IFN-γ-secreting spots and higher levels of IL-5, a cytokine characteristic of T_{H}2-biased CD4+ T-cell responses. See FIG. 1B.

### Example 3

### Use of MVA-BN as an Adjuvant in Combination with Recombinant HER2 Protein Confers Anti-Tumor Efficacy in Mice Challenged with CT26-HER-2-Expressing Tumor Cells

BALB/c mice (n=9) were challenged intravenously (i.v.) with 5×10⁵ CT26-HER-2 cells on day 1 and treated intraperitoneally (i.p.) on day 4 with (1) TBS; (2) 5×10⁷ IU MVA-BN−HER2; (3) live MVA-BN+7.5 μg HER2 protein; (4) killed MVA-BN+7.5 μg HER2 protein; (5) live MVA-BN; or (6) killed MVA-BN. Viruses were killed by heat inactivation. Lung weights were determined on day 15. Statistics: One Way ANOVA with Bonferroni adjustment.

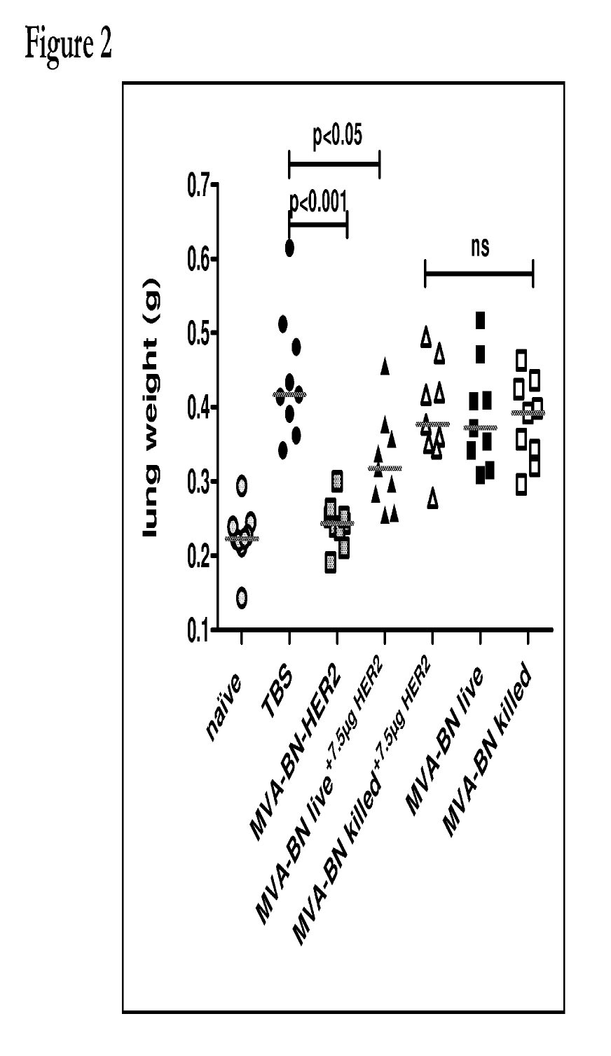

FIG. 2 shows that statistically-significant anti-tumor activity requires the presence of both live virus and HER2 protein (i.e., MVA-BN+HER2) or virus expressing the HER2 transgene MVA-BN−HER2).

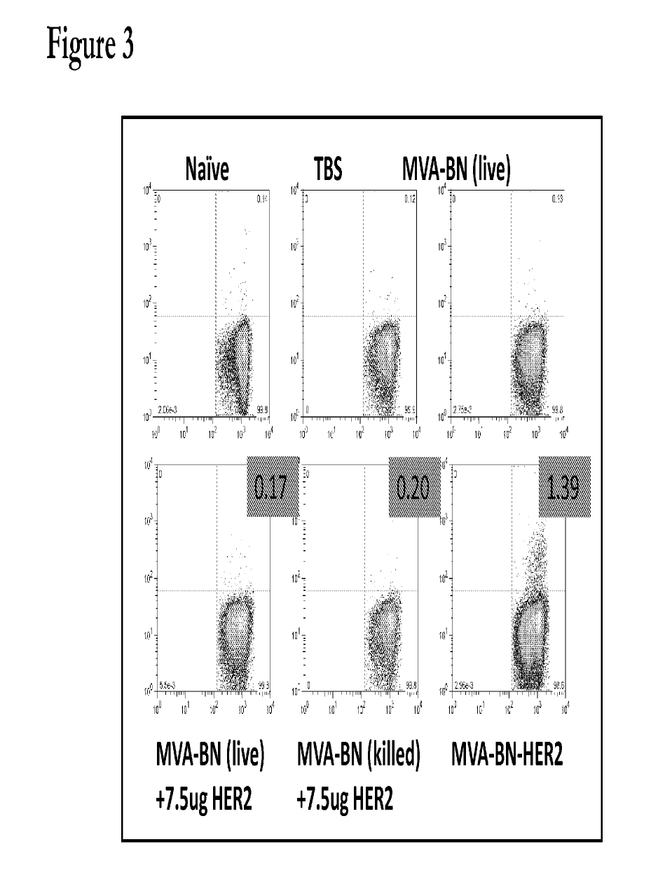

CD8+ T-cells isolated from lungs of vaccinated mice were analyzed by fluorescence-activated cell sorting (“FACS”) HER-2-specific CD8⁺ T-cells were detected with an H-2 K^{d} pentamer loaded with the p63 peptide NH₂-TYLPTNASL-COOH according to the manufacturer's instructions and standard procedures (ProImmune, Oxford, UK). FIG. 3 shows that only treatment with MVA-BN−HER2 induces HER-2 specific CD8+ T-cells in the tumor. FIG. 3 also shows that induction of HER-2-specific CD8+ T-cells requires expression of HER-2 from the viral vector; addition of recombinant HER2 protein is not sufficient.

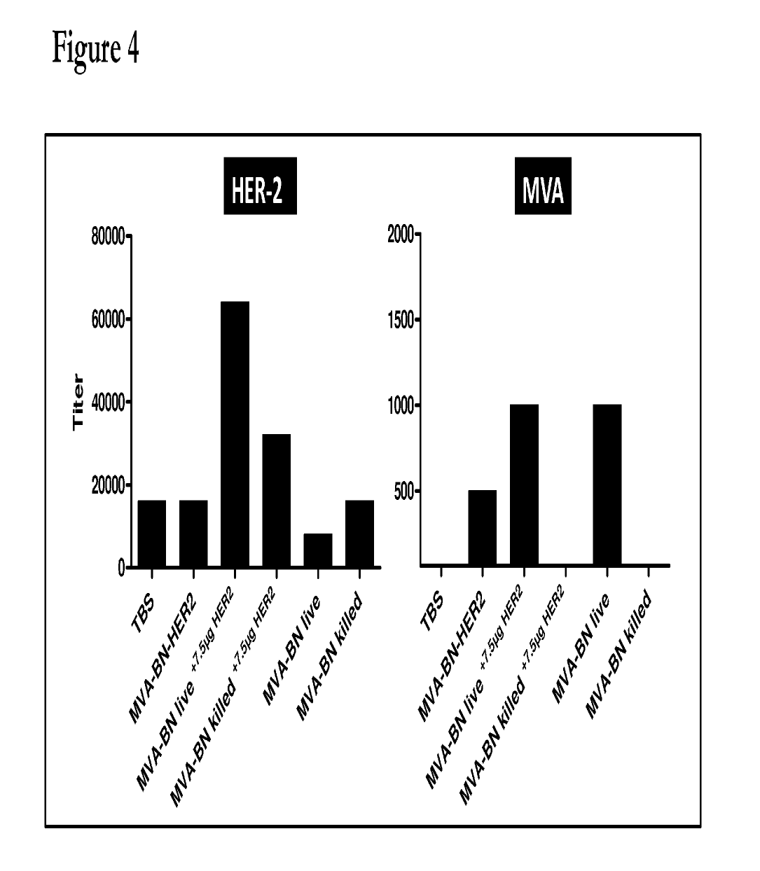

Finally, IgG antibody titers specific for HER-2 or MVA-BN were determined by ELISA on pooled serum from mice challenged with CT26-HER-2 and treated as described above. FIG. 4 shows that anti-tumor efficacy induced by use of MVA-BN as an adjuvant may be mediated by HER-2-specific antibodies.

### Example 4

### Addition of HER2 Protein to MVA-BN−HER-2 Significantly Increases Anti-HER-2 Antibody Titers

BALB/c mice (n=5) were vaccinated s.c. every two weeks for four cycles (i.e., a total treatment time of eight weeks) with: (1) TBS; (2) 1×10⁷ IU MVA-BN−HER2; or (3) 1×10⁷ IU MVA-BN−HER2+5 μg recombinant HER2 protein. Total IgG antibody titers or isotype-specific titers (e.g., IgG2a and IgG1) were determined on pooled serum by ELISA according to standard methods as described in Example 1.

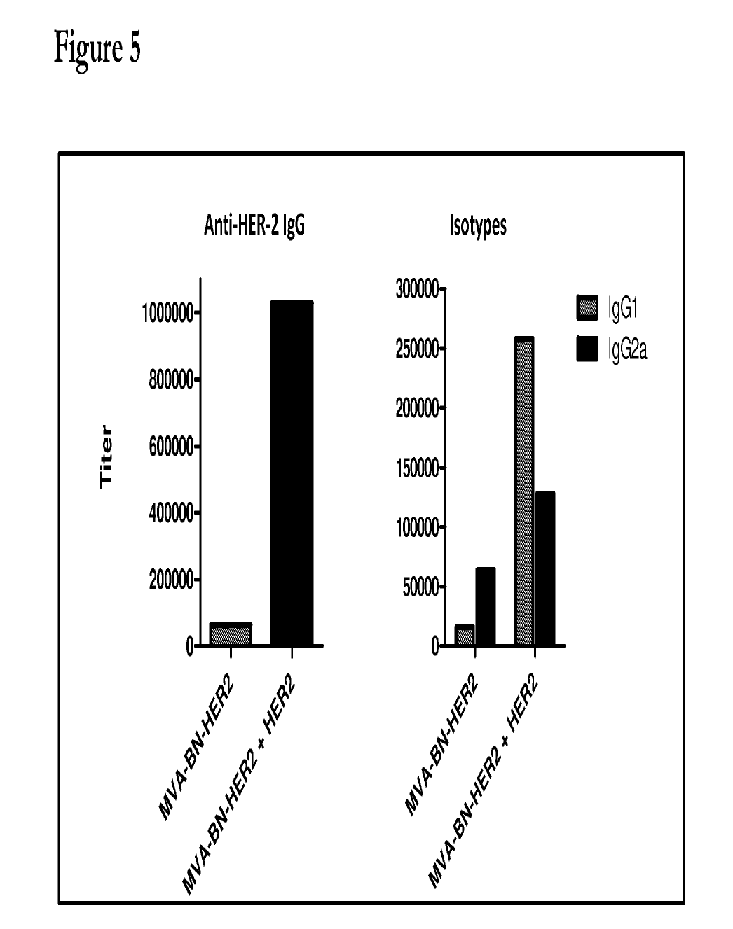

FIG. 5 shows that HER-2-specific antibody titers significantly increased with addition of HER2 protein, and that the titer of isotype IgG1 increased proportionally more than the titer of isotype IgG2a. This suggests that the HER2 protein adds a different quality to the immune response elicited by the recombinant MVA vector. This response may still be T_{H}1-biased overall, but includes some T_{H}2 qualities that could be beneficial for the treatment of cancer.

### Example 5

### Addition of HER2 Protein Increases HER-2-Specific CD4+ But Not CD8+ T-Cell Responses

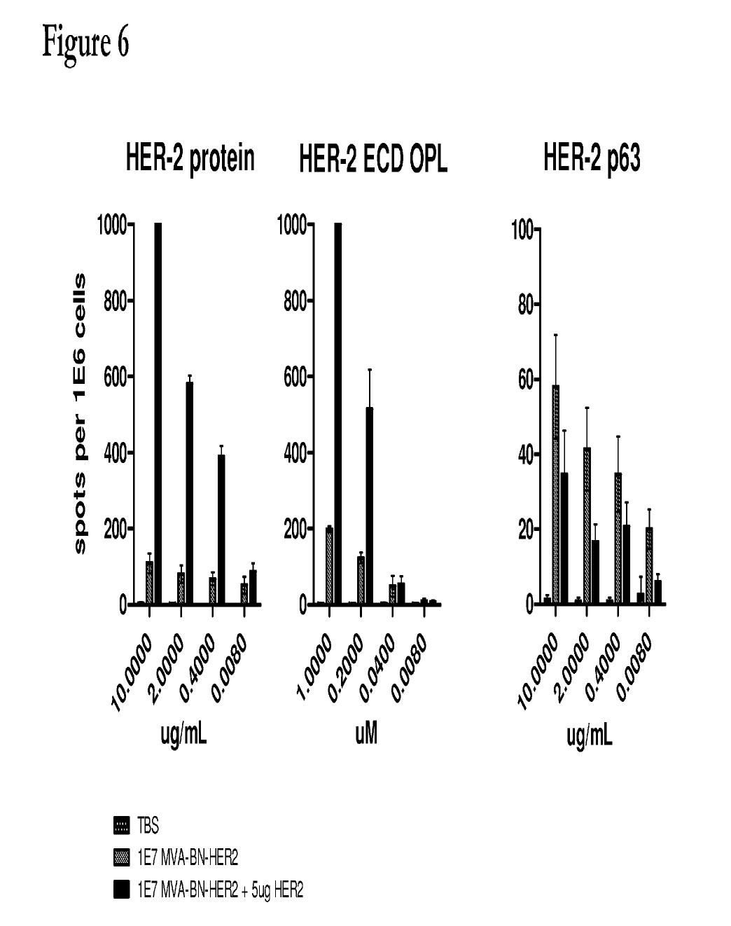

BALB/c mice (n=5) were treated s.c. every two weeks for four cycles (i.e., a total treatment time of eight weeks) with: (1) TBS; (2) 1×10⁷ IU MVA-BN−HER2; or (3) 1×10⁷ IU MVA-BN−HER2+5 μg recombinant HER2 protein. Two weeks after the final treatment, the frequencies of responding T-cells in splenocytes were determined by the IFN-γ ELISPOT assay. Splenocytes were restimulated with HER-2 protein, a HER-2 overlapping peptide library (“OPL”), or the K^{b}-restricted CD8+ peptide (HER-2 p63). FIG. 6 shows that the addition of recombinant HER2 protein increased CD4+ T-cell responses but not CD8+ T-cell responses. In addition, depletion experiments demonstrated that responses to HER-2 protein or HER-2 OPL are CD4+ T-cell-specific (data not shown).

### Example 6

### Improved Active Immunotherapy for the Treatment of Breast Cancer in a Mouse Model

TUBO cells are a cloned cell line established in vitro from a lobular carcinoma that arose spontaneously in a BALB/c-NeuT mouse(Bovero et al., J. Immunol. 165:5133-5142 (2000)). In contrast to many other tumor models, the mechanism of protection against this Erb-2-expressing tumor is known to be mainly antibody-mediated (Park et al., Cancer Res. 68(6):1979-1987 (2008)). Curcio C., et al. (J. Clin. Invest. 111(8):1161-1170 (2003)) performed immunotherapy of TUBO cells in wild-type (“WT”) or knockout (“KO”) BALB/c mice. In an immunotherapy setting, the ability to generate an effective antitumor attack that overcomes the kinetics of tumor proliferation and tumor escape mechanisms appears to result from the combination of multiple mechanisms, in which Abs were able to efficiently control growth of TUBO tumors. Additional effector mechanisms include FcγRI/III-mediated antibody-dependent cell-mediated cytotoxicity (“ADCC”), perforin, IFN-γ, Natural Killer (“NK”) cells and macrophages.

This experiment was performed to determine if the addition of HER2 protein to vaccination with MVA-BN−HER2 improves efficacy in the TUBO tumor model.

Vaccination Protocol 40 BALB/c males, 7-8 weeks old (DOB Jan. 5, 2010) were randomized into four groups (n=10). Group 1 was vaccinated with Tris-Buffered Saline (10 mM TRIS, 140 mM NaCl, pH 7.7; “TBS”) without recombinant HER2 protein; Group 2 was vaccinated with 5×10⁷ infectious units (“IU”) of MVA-BN−HER2 without recombinant HER2 protein; Group 3 was vaccinated with 5×10⁷ IU of MVA-BN−HER2 plus 5 μg recombinant HER2 protein; and Group 4 was vaccinated with 5 μg recombinant HER2 protein formulated in Complete Freund's Adjuvant (“CFA”) on day 1 and Incomplete Freund's Adjuvant (“IFA”) on day 15. Vaccinations were administered on day 1 and day 15 of the study; vaccines were administered by subcutaneous injection at two sites, and each injection was delivered in a volume of 200 μl, except HER2 formulated in CFA or IFA was delivered at a single site in a volume of 100 μl. Mice were challenged with a single injection 1×10⁵ TUBO tumor cells administered intradermally.

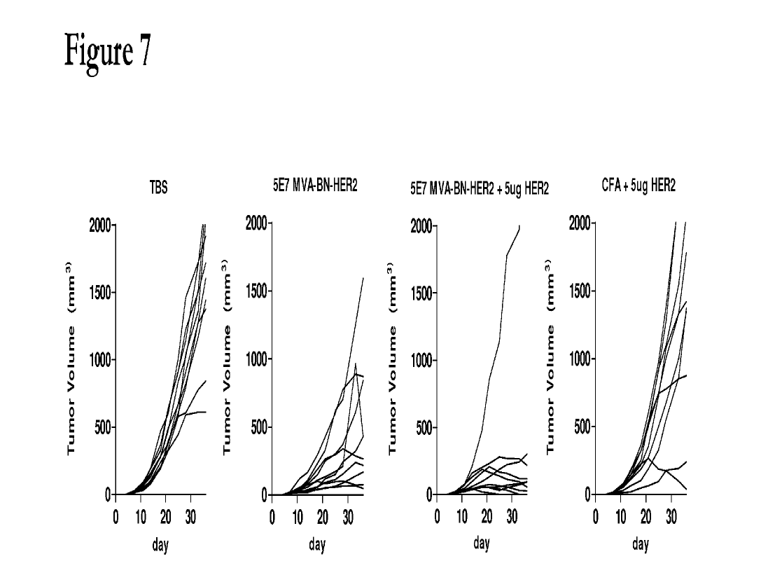

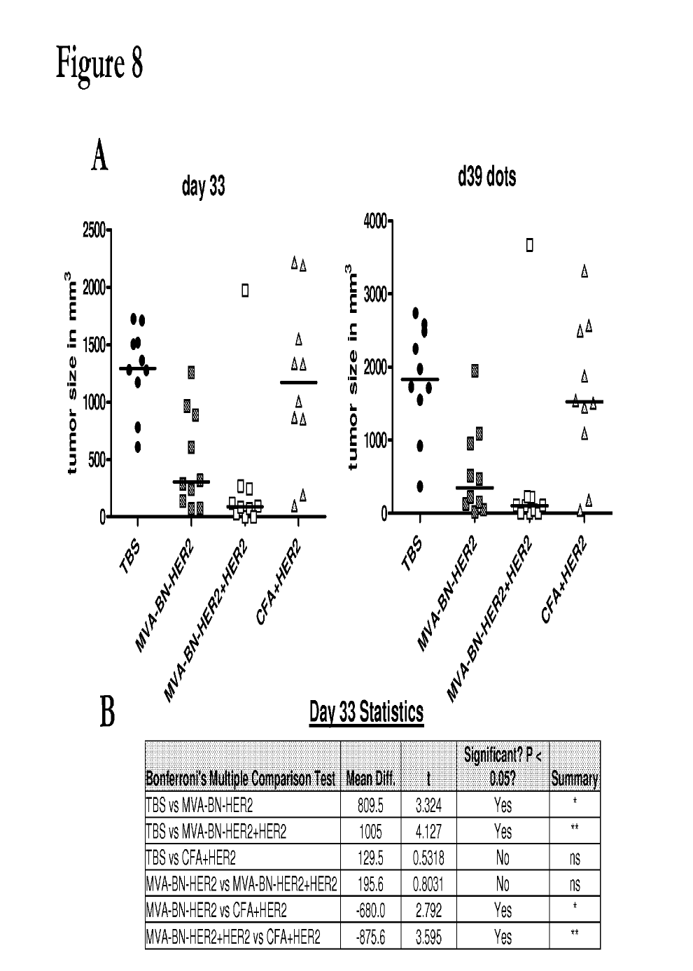

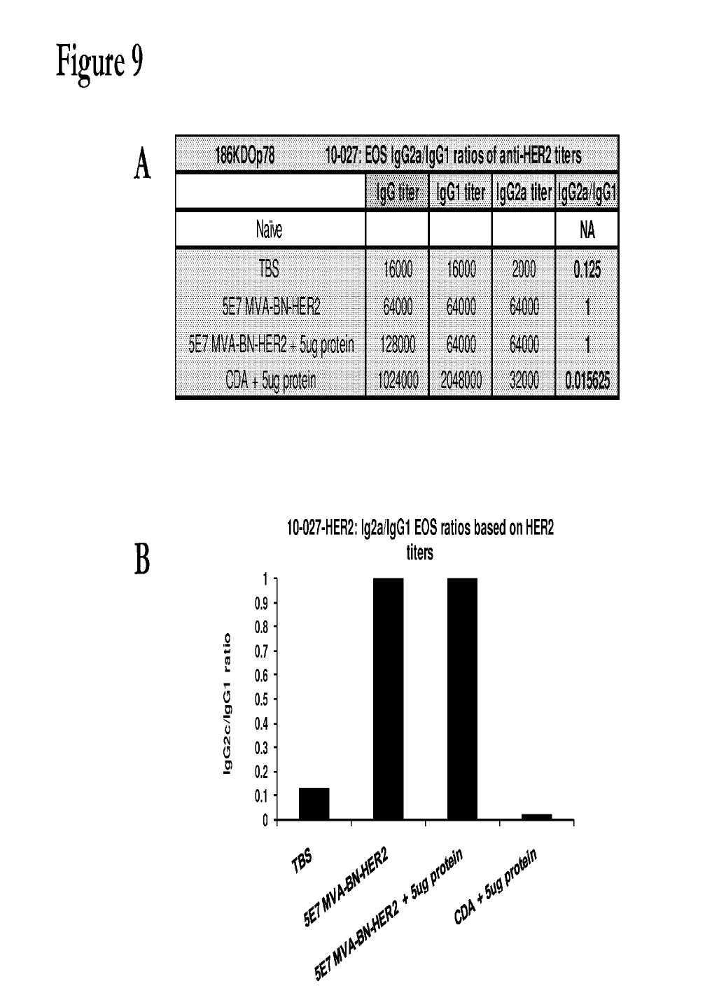

Results: Anti-tumor efficacy. Immunotherapy with MVA-BN®−HER2 resulted in significant delay of tumor growth (p<0.05). Immunotherapy with MVA-BN®−HER2+HER2 also resulted in significant delay of tumor growth (p<0.01). Two mice were tumor free on day 39 post-treatment. Addition of HER2 protein showed a trend of improved protection as demonstrated by increased significance (improved from p<0.05 for MVA-BN alone to p<0.01 for MVA-BN−HER2+HER2) compared to the TBS group. This suggests that adding HER2 protein improved anti-tumor efficacy, although this did not reach statistical significance when the two groups were compared to each other. Treatment with HER2 protein formulated in CFA overall did not show significant anti-tumor efficacy, although 2 out of 10 mice rejected the TUBO tumors. See FIGS. 7 and 8.

Results: Antibody responses. ELISAs were performed according to the standard protocol presented in Example 1. Titers were reproducible in 2 separate determinations. Tumor-induced titers were 16000. Treatment with MVA-BN®−HER2 increased titers 4-fold to 64000, while addition of protein only increased titer by 2-fold over MVA-BN®−HER2 alone, to 128000. Titers induced by CFA+HER2 were 10-to 20-fold higher (1-2000000). As usual, tumor-and CFA formulation-induced Ab were highly T_{H}2-biased, whereas MVA-BN®−HER2-induced Ab were T_{H}1-biased, and addition of protein to MVA-BN®−HER2 did not change the isotype ratio. This is the second tumor model demonstrating that high titers of IgG₁ antibodies do not result in anti-tumor efficacy in BALB/c mice; the other being the CT26-HER2 ELM model.

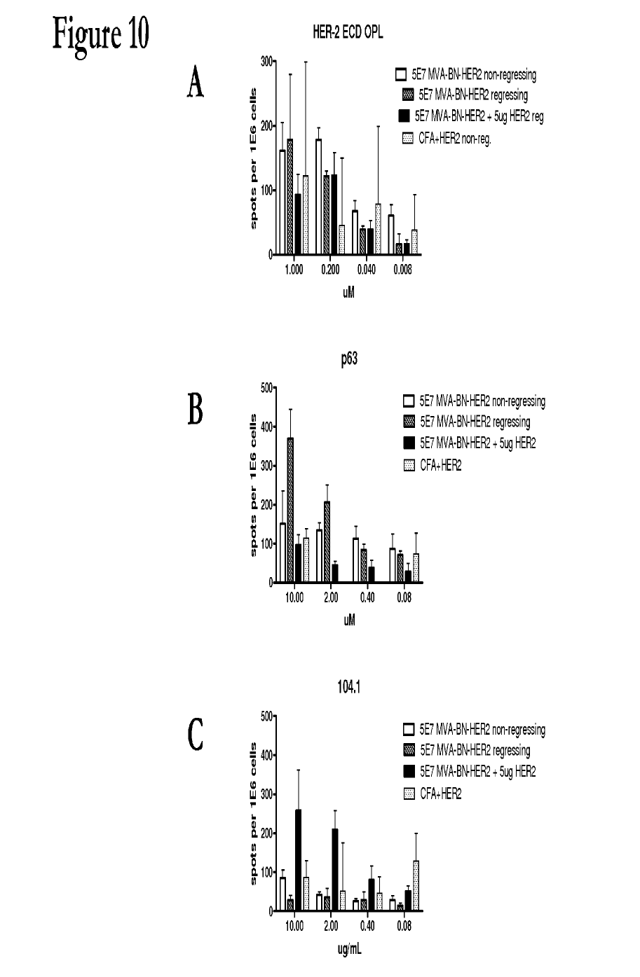

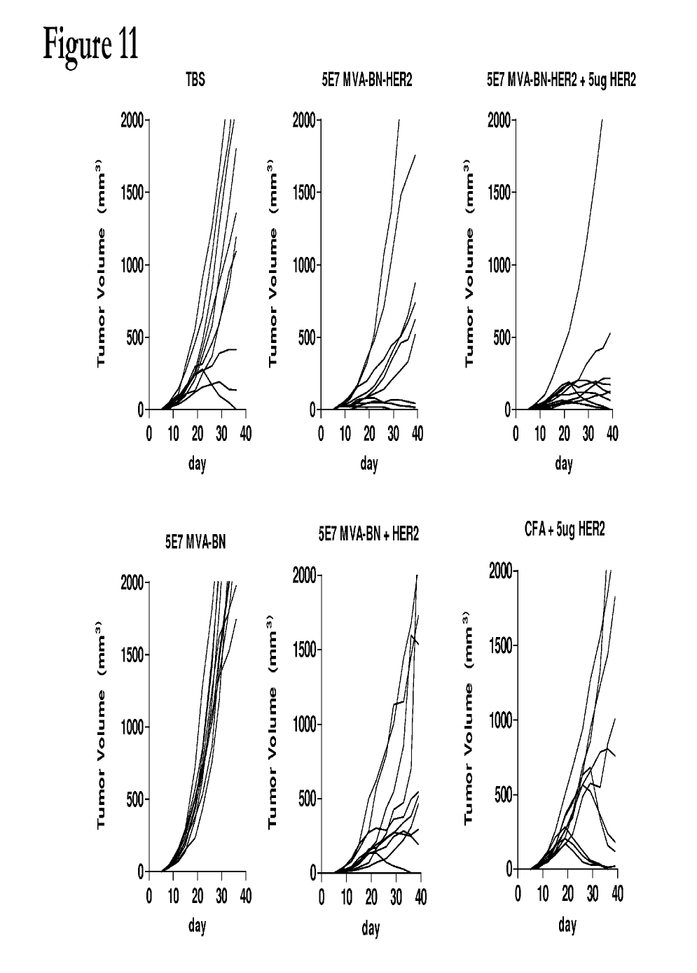

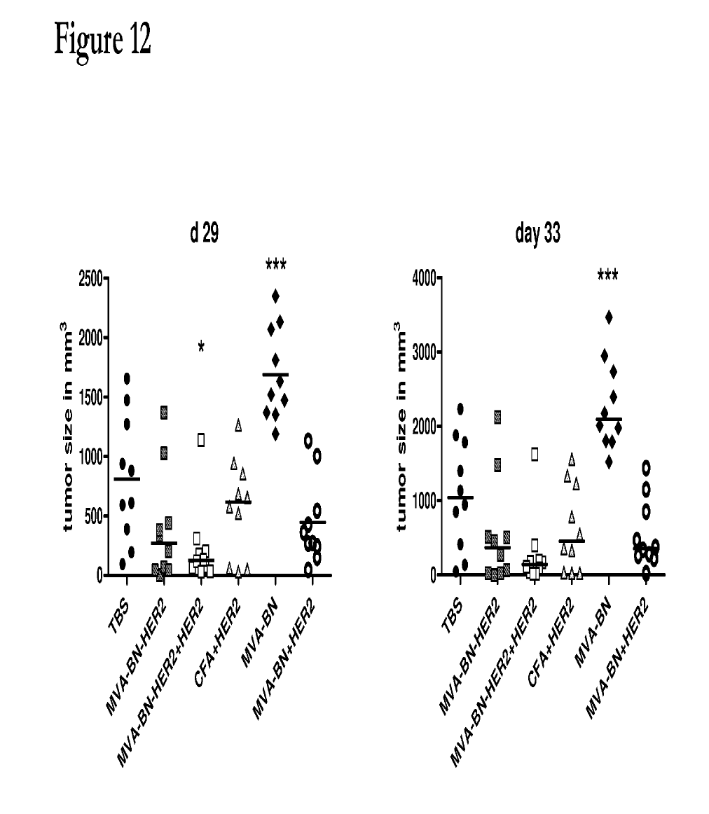

Results: T-cell responses. Mice in the MVA-BN®−HER2 group were divided into tumor regressing and tumor non-regressing mice. All tumors of mice in the MVA-BN®−HER2+HER2 group were regressing; none of the tumors in the CFA+HER2 group were regressing. ELISPOT assays were performed according to the standard protocol presented in Example 1 on day 41 post-tumor implantation. As in other experiments, addition of HER2 protein to MVA-BN®−HER2 boosted the HER2 protein response as measured via ELISPOT using 104.1 protein, the HER2 extracellular domain modified to incorporate two T_{H} epitopes derived from tetanus toxin (see FIG. 4C), but not when measured using p63, a CD8 epitope derived from HER-2 that is active in BALB/c mice (see FIG. 10B). Interestingly, in mice that were treated with MVA-BN®−HER2 and rejected the tumor, a strong p63 response was noticeable. The HER2 ECD overlapping peptide library (“OPL”) response was similar in all groups (see FIG. 10A), suggesting the possibility that different effector mechanisms may be responsible for the observed effect in regressing tumors from MVA-BN®−HER2 compared to MVA-BN®−HER2+HER2 treated animals. See FIG. 10.

### Example 7

### Improved Active Immunotherapy for the Treatment of Breast Cancer in a Mouse Model

This experiment was performed to determine if the addition of HER2 protein to vaccination with MVA-BN or MVA-BN−HER2 improves efficacy in the TUBO tumor model.

Vaccination Protocol. 60 BALB/c females, 7-8 weeks old (DOB Mar. 5, 2010, and Mar. 12, 2010) were randomized into 6 groups (n=10). Group 1 was vaccinated with TBS; Group 2 was vaccinated with 5×10⁷ IU MVA-BN−HER2 without recombinant HER2 protein; Group 3 was vaccinated with 5×10⁷ IU MVA-BN−HER2+5 μg recombinant HER2 protein; Group 4 was vaccinated with 5×10⁷ IU MVA-BN without recombinant HER2 protein; Group 5 was vaccinated with 5×10⁷ IU MVA-BN+5 μg recombinant HER2 protein; and Group 6 was vaccinated with 5 μg recombinant HER2 protein formulated in CFA on day 1 or Incomplete Freund's Adjuvant (“IFA”) on day 15. Vaccinations were administered on day 1 and day 15 of the study; vaccines were administered by subcutaneous injection at two sites, and each injection was delivered in a volume of 200 μl except for the recombinant protein formulated in CFA or IFA, which was delivered in a volume of 100 μl. Mice were challenged with a single injection 1×10⁵ TUBO tumor cells administered intradermally.

Results: Anti-tumor efficacy. In this experiment, statistically significant anti-tumor efficacy was only reached transiently (days 12 through 22 by Student's t test, but not by ANOVA with Bonferroni adjustment) after MVA-BN−HER2 treatment alone, although anti-tumor efficacy was achieved to a statistically significant level in the prior experiment (p<0.05 by ANOVA with the Bonferroni adjustment). However, treatment with MVA-BN−HER2+HER2 did result in statistically significant protection on day 29 (p<0.05 by ANOVA, compared to p<0.01 in the prior study). Statistically significant protection (assessed by Student's t test) was reached in this group from days 12 to 36, when the control group treated with TBS started to die, demonstrating a clear benefit from adding HER2 protein to MVA-BN−HER2 treatment. No other treatment induced significant protection as measured by tumor size, however addition of HER2 protein to MVA-BN or formulated in CFA did show some tumor regression at later time points, possibly due to Ab-mediated mechanisms. Treatment with MVA-BN significantly increased tumor burden in this experiment. This observation was not repeated in subsequent experiments and appears to be a spurious result.

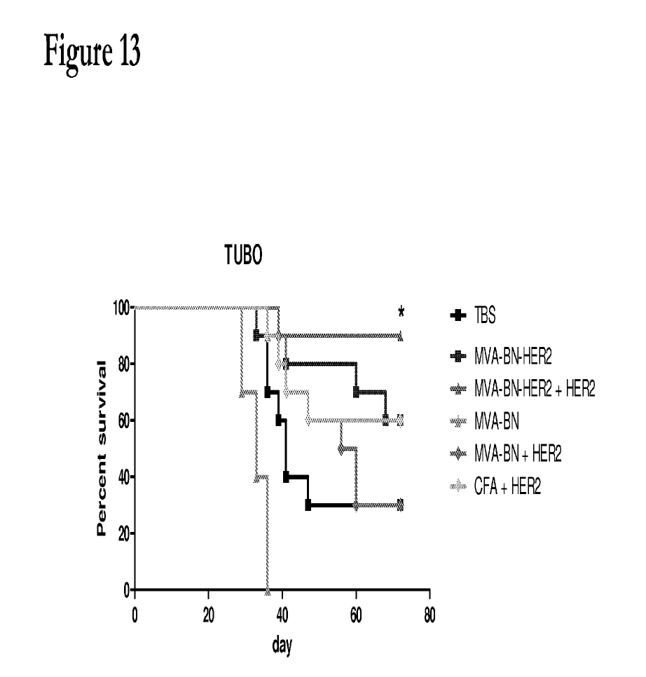

Results: Survival analysis. Median survival of the control group receiving TBS was 41 days. Treatment with MVA-BN alone decreased median survival to 33 days. As mentioned previously, this observation was not repeated in subsequent experiments and appears to be a spurious result. Treatment with MVA-BN−HER2 or CFA+HER-2 provided 60% survival by the conclusion of the experiment on day 68. Treatment with MVA-BN+HER-2 increased the median survival from 41 to 58 days but did not increase the survival rate as compared to the TBS group. Treatment with MVA-BN−HER2+HER-2 resulted in 90% survival by day 68. 3 out of 10 mice were tumor free at that time point. This is statistically significant by Log-rank test p=0.008. The Log-rank test was used in preparing the Kaplan-Maier curve. See FIG. 13.

Results: Antibody responses. ELISAs were performed according to the standard protocol presented in Example 1. Data are not as clean cut as in the study described in Example 6, likely because serum was collected much later in the present study, and was not ‘synchronized’ as in the previous study. As expected, MVA titers in all groups were very similar, in the range between 8000 and 16000. Addition of recombinant HER2 protein had no effect on the IgG isotype ratios of anti-MVA antibodies.

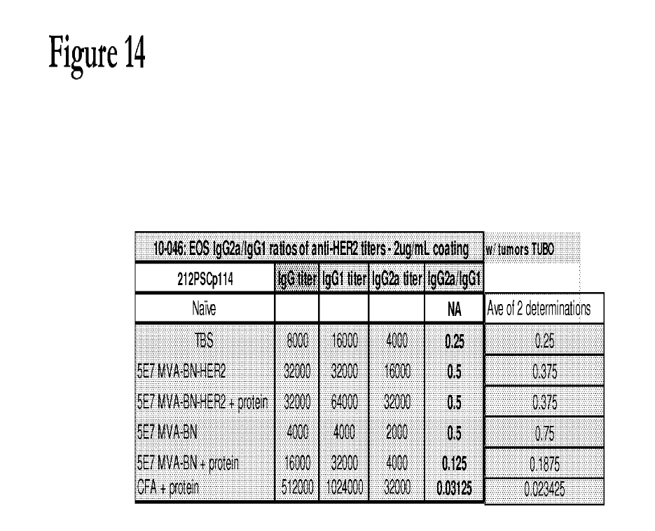

Treatment with MVA-BN+HER2, MVA-BN−HER2 or MVA-BN−HER2+HER2 induced similar HER-2 titers that were 2-4-fold higher than tumor-induced HER-2 titers. See FIG. 14. No significant changes in the IgG isotype ratios were observed, although a trend of higher IgG2a ratios was noted in groups treated with MVA-BN−HER2 or MVA-BN−HER2+HER2. Mice treated with MVA-BN had HER-2 Ab titers 4-fold lower than mice receiving TBS alone or mice treated with MVA-BN+HER2. This could be due to the fact that those mice had to be taken down earlier than the other groups. Mice treated with CFA had by far the highest HER-2 titers; indeed, those titers were up to 100-fold higher than other groups, and the response was mainly a T_{H}2 isotype (IgG1).

### Example 8

### Improved Active Immunotherapy for the Treatment of Breast Cancer in a Mouse Model

This experiment was performed to determine if the addition of HER2 protein to vaccination with MVA-BN or MVA-BN−HER2 improves efficacy in the TUBO tumor model.

Vaccination Protocol. 60 BALB/c females, 7-8 weeks old (DOB May 20, 2010) were randomized into 6 groups (n=10). Group 1 was vaccinated with TBS; Group 2 was vaccinated with 5×10⁷ IU MVA-BN−HER2 without recombinant HER2 protein; Group 3 was vaccinated with 5×10⁷ IU MVA-BN−HER2+5 μg recombinant HER2 protein; Group 4 was vaccinated with 5×10⁷ IU MVA-BN without recombinant HER2 protein; Group 5 was vaccinated with 5×10⁷ IU MVA-BN+5 μg recombinant HER2 protein; and Group 6 was vaccinated with 5 μg recombinant HER2 protein formulated in CFA (day 1) or IFA (day 15). Vaccinations were administered on day 1 and day 15 of the study; vaccines were administered by subcutaneous injection at two sites, and each injection delivered in a volume of 200 μl except for the recombinant HER2 protein formulated in CFA or IFA, which was delivered in a volume of 100 μl. Mice were challenged with a single injection 1×10⁵ TUBO tumor cells administered intradermally. Immune response was monitored by ELISA and ELISPOT performed after tumor challenge. ELISPOT assays were performed on day 55 post-implantation.

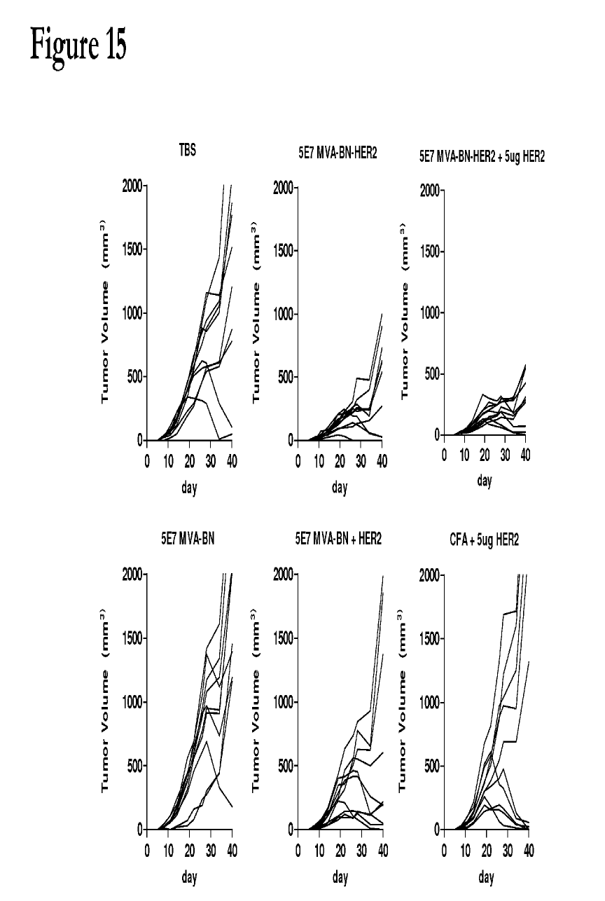

Results: Anti-tumor efficacy. In this experiment, some TUBO tumors regressed in the TBS control group starting around day 25 after implantation. Groups treated with MVA-BN−HER2 and MVA-BN−HER2+HER-2 both showed significant anti-tumor activity. These two groups were not significantly different from each other, but were significantly different from treatment with MVA-BN alone or CFA+HER-2. MVA-BN+HER2 did show some significant anti-tumor efficacy as measured by Student's t test, but this did not reach statistical significance compared to the group receiving TBS only as measured by ANOVA with Bonferroni adjustment. At later time points, tumor regression occurred in all groups and statistical significance was lost. 5 out of 10 mice in the CFA+HER-2 treatment group were tumor-free suggesting that IgG1 antibodies can confer anti-tumor efficacy at late timepoints in this model. See FIG. 15.

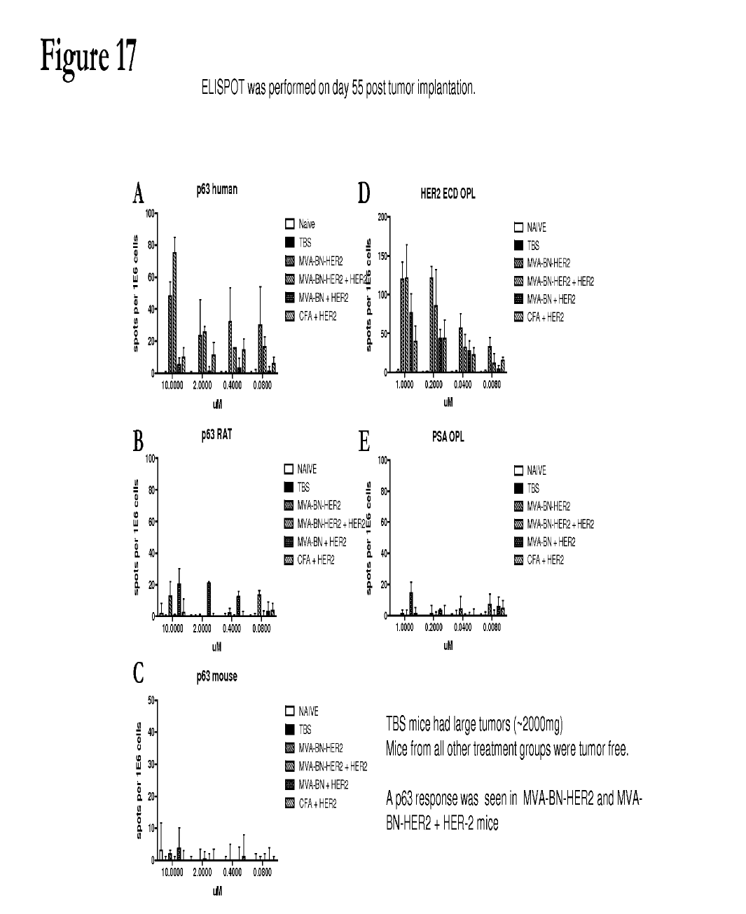

Results: T-cell responses. ELISPOT assays were performed according to the standard protocol presented in Example 6. ConA responses were similar in all but the TBS group, which may reflect immunosuppressive activities of the growing tumor. All mice that were treated with MVA had a significant and-MVA response. However, responses varied in magnitude by 2-3-fold, especially after restimulation with the F2L peptide. MVA-BN−HER2 and MVA-BN−HER2+5 μg recombinant HER2 protein showed significant p63 responses (60-80 spots). HER-2-specific responses were lower in MVA+HER2 and CFA+HER2 treated mice, especially with respect to p63 responses. See FIG. 17. Overall HER2 responses were lower than in the study described in Example 6. This is within the normal inter-assay variation range for ELISPOT assays. This assay also tested cross-reactivity between human, mouse and rat p63. Mice immunized with human HER2 and carrying rat HER2-expressing tumors did not have significant levels of rat p63-specific T cells, an observation in contrast to a report by Jacob et al. (Cell. Immunol., 240:96-106 (2006)), in which DNA immunization induced low levels of cross-reactive T-cells in tumor-free BALB/c mice. Unlike the present studies, however, the ELISPOT assays reported by Jacobs restimulated with antigen-presenting cells (“APC”) loaded with 100 μg of peptide per ml, rather than with peptide alone, so the significance of Jacobs' findings remains unclear.

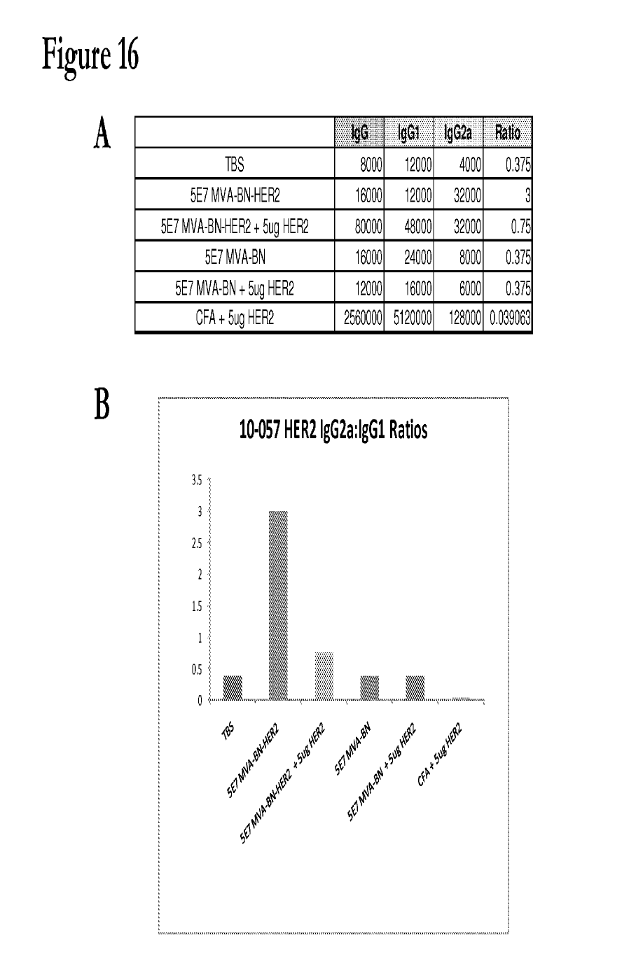

Results: Antibody responses. ELISAs were performed according to the standard protocol presented in Example 1. Data was not as clean cut as in the study described in Example 6, likely because serum was collected much later in the present study, and collection was not ‘synchronized’ as in the previous study. MVA titers were the same in all treatment groups. As expected, addition of HER2 protein did not affect the MVA titers or the ratio of IgG2a to IgG1. The growing TUBO tumors induced HER-2 titers of ˜8000 and IgG2a to IgG1 ratios of 0.375. Similar to Example 6, the HER2 titers were modestly different between the groups. However, treatment with HER-2 formulated in CFA also shifted the IgG2a:IgG1 ratio nearly 100-fold further towards a T_{H}2 response compared to treatment with MVA-BN−HER2, producing an IgG2a:IgG1 ratio of 0.039. See FIG. 16.

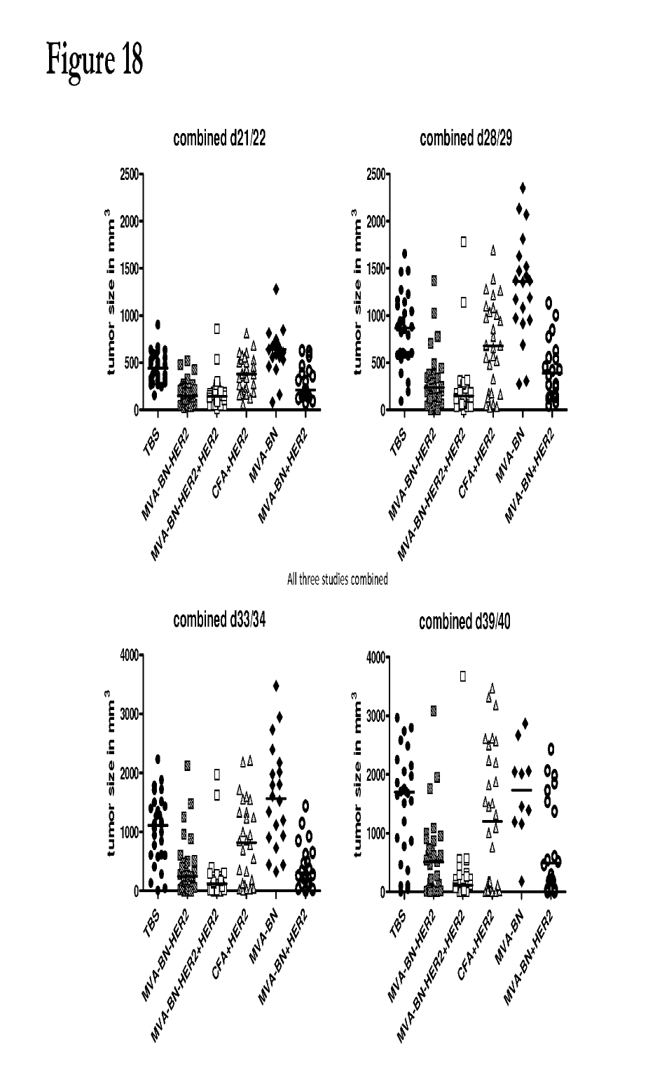

Conclusions. To further characterize the mechanism of action of MVA-BN®−HER2 the immunologic function of the MVA-BN® vector as an adjuvant was explored. Preclinical experiments comparing the immune responses and anti-tumor efficacy of MVA-BN® or MVA-BN®−HER2 as adjuvants when mixed with recombinant HER2 protein demonstrated that MVA-BN® has potent adjuvant activity and requires live virus. HER-2 specific immune responses and anti-tumor efficacy were induced; however, expressing the HER2 protein directly from the vector as in MVA-BN®−HER2 was superior. Addition of protein to MVA-BN®−HER2 further increased HER-2 specific immune responses particularly with respect to CD4 T cell and antibody responses. This resulted in improved anti-tumor efficacy in the TUBO breast cancer model. This suggests that mixing the recombinant HER2 protein with MVA-BN−HER2 results in a qualitatively different immune response compared to immunization with recombinant HER2 protein formulated in PBS or CFA, or MVA-BN−HER2 alone. These data showed that the anti-tumor activity of MVA-BN®−HER2 could potentially be increased by adding HER2 protein to the recombinant vector. See FIG. 18. And although the antibody response was not quantitatively different when in the presence or absence of recombinant HER2 protein, there are a number of ways in which the quality of the immune response might vary that could account for the difference including, for example, increased ADCC activity, increased secretion of perforin or IFN-γ, or increased NK cell and/or macrophage activity.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A composition comprising a recombinant modified vaccinia virus Ankara (“MVA”) expressing a polypeptide comprising a heterologous tumor-associated antigen and a recombinant protein comprising the heterologous tumor-associated antigen.

2. The composition of claim 1, wherein the heterologous tumor-associated antigen comprises an antigen selected from the group consisting of a c-erbB-1 (“EGFr”) antigen, a c-erbB-2 (“HER-2” or “c-Neu”) antigen, a c-erbB-3 antigen and a c-erbB-4 antigen.

3. The composition of claim 2, wherein the heterologous tumor-associated antigen comprises a HER-2 antigen.

4. The composition of claim 3, wherein the HER-2 antigen comprises SEQ ID NO:1.

5. The composition of claim 4, wherein the MVA is MVA-Bavarian Nordic (“MVA-BN”).

6. The composition of claim 5, wherein administration of the composition induces an improved antigen-specific CD4+ T-cell response as measured by ELISPOT assay or increased antigen-specific antibody titers as measured by ELISA, compared to administration of a composition comprising only a recombinant MVA expressing a polypeptide comprising a heterologous tumor-associated antigen.

7. The composition of claim 6, further comprising one or more pharmaceutically acceptable diluents, buffers, excipients, or carriers.

8. A method of treating a human cancer patient comprising: (a) identifying a human cancer patient having a cancer expressing a tumor-associated antigen; (b) administering to the patient a therapeutically effective amount of a recombinant modified vaccinia virus Ankara (“MVA”) expressing a polypeptide comprising the heterologous tumor-associated antigen and a therapeutically effective amount of a recombinant protein comprising the heterologous tumor-associated antigen, thereby treating the cancer.

9. The method of claim 8, wherein the heterologous tumor-associated antigen comprises an antigen selected from the group consisting of a c-erbB-1 (“EGFr”) antigen, a c-erbB-2 (“HER-2” or “c-Neu”) antigen, a c-erbB-3 antigen and a c-erbB-4 antigen.

10. The method of claim 9, wherein the heterologous tumor-associated antigen comprises a HER-2 antigen.

11. The method of claim 10, wherein the HER-2 antigen comprises SEQ ID NO:1.

12. The method of claim 11, wherein the MVA is MVA-BN.

13. The method of claim 12, wherein administration of the recombinant MVA and the recombinant protein together induces an improved antigen-specific CD4+ T-cell response as measured by ELISPOT assay or increased antigen-specific antibody titers as measured by ELISA, compared to administration of a composition comprising only a recombinant MVA expressing a polypeptide comprising a heterologous tumor-associated antigen.

14. The method of claim 13, wherein the recombinant MVA and the recombinant protein are formulated with one or more pharmaceutically acceptable diluents, buffers, excipients, or carriers.

15. The method of claim 14, wherein the human cancer patient has a HER-2 over-expressing cancer.

16. The method of claim 15, wherein the HER-2 over-expressing cancer is selected from the group consisting of breast cancer, colon cancer, lung cancer, gastric cancer, pancreatic cancer, bladder cancer, cervical cancer and ovarian cancer.

17. The method of claim 16, wherein the HER-2 over-expressing cancer is breast cancer.

18. The method of claim 17, wherein the breast cancer is metastatic breast cancer.

19. A kit for treating a human cancer patient, comprising: (a) a recombinant modified vaccinia virus Ankara (“MVA”) expressing a polypeptide comprising a heterologous tumor-associated antigen; (b) a recombinant protein comprising the heterologous tumor-associated antigen; and (c) instructions to administer a therapeutically effective amount of the recombinant MVA and a therapeutically effective amount of the recombinant protein to a human cancer patient having a cancer expressing the heterologous tumor-associated antigen.

20. The kit of claim 19, wherein the heterologous tumor-associated antigen comprises an antigen selected from the group consisting of a c-erbB-1 (“EGFr”) antigen, a c-erbB-2 (“HER-2” or “c-Neu”) antigen, a c-erbB-3 antigen and a c-erbB-4 antigen.

21. The kit of claim 20, wherein the heterologous tumor-associated antigen comprises a HER-2 antigen.

22. The kit of claim 21, wherein the HER-2 antigen comprises SEQ ID NO:1.

23. The kit of claim 22, wherein the MVA is MVA-BN.

24. The kit of claim 23, wherein administration of the recombinant MVA and the recombinant protein together induces an improved antigen-specific CD4+ T-cell response as measured by ELISPOT assay or increased antigen-specific antibody titers as measured by ELISA, compared to administration of a composition comprising only a recombinant MVA expressing a polypeptide comprising a heterologous tumor-associated antigen.

25. The kit of claim 24, wherein the recombinant MVA and the recombinant protein are formulated with one or more pharmaceutically acceptable diluents, buffers, excipients, or carriers.

26. The kit of claim 25, wherein the human cancer patient has a HER-2 over-expressing cancer.

27. The kit of claim 26, wherein the HER-2 over-expressing cancer is selected from the group consisting of breast cancer, colon cancer, lung cancer, gastric cancer, pancreatic cancer, bladder cancer, cervical cancer and ovarian cancer.

28. The kit of claim 27, wherein the HER-2 over-expressing cancer is a breast cancer.

29. The kit of claim 28, wherein the breast cancer is metastatic breast cancer.


SEQUENCE LISTING (2 sequences)

SEQ ID NO: 1 - protein, 683 aa, Artificial
  Human HER2 protein modified to contain two T-helper cell epitopes derived from Tetanus toxin
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVQYIKANSKFIGITELRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSFNNFTVSFWLRVPKVSASHLEIVSAVVGIL

SEQ ID NO: 2 - DNA, 2052 bp, Artificial
  A cDNA sequence encoding the modified HER2 protein of SEQ ID NO1
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcttaagtttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtccagtacatcaaagctaactccaaattcatcggtatcaccgagctgcggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccttcaacaacttcaccgtgagcttctggctgcgcgtgcccaaggtgagcgccagccacctggagatcgtctctgcggtggttggcattctgtag